(19) United States
(12) Patent Application Publication
(10) Pub. No.: US 2025/0064324 A1
(43) Pub. Date: Feb. 27, 2025

(54) SYSTEMS AND METHODS FOR MONITORING AND MANAGING SLEEP

(71) Applicant: ResMed Pty Ltd, Bella Vista, New South Wales (AU)

(72) Inventors: Alberto ZAFFARONI, Dublin (IE); Ramyabai GUNALAN, Singapore (SG); Gregory KLUENDER, Bremen (DE); Kayla Jade DOLORES, Syndney (AU); Hui Leng GAN, Singapore (SG); Bao Hui LEE, Singapore (SG); Lyndsay Brookes TOLL, San Diego, CA (US)

(21) Appl. No.: 18/723,302

(22) PCT Filed: Dec. 21, 2022
(86) PCT No.: PCT/AU2022/051547

§ 371 Date: Jun. 24, 2024

(30) Foreign Application Priority Data
Dec. 23, 2021 (AU) 2021904232
Jul. 15, 2022 (AU) 2022901994

(51) Int. Cl. A61B 5/0205; A61B 5/00; A61B 5/024; A61B 5/026; A61B 5/145; A61B 5/28; G16H 10/20; G16H 10/60; G16H 20/30; G16H 80/00

(52) U.S. Cl. CPC A61B 5/0205; A61B 5/0002; A61B 5/4809; A61B 5/4815; A61B 5/4818; A61B 5/6801; A61B 5/7275; G16H 10/20; G16H 10/60; G16H 20/30; G16H 80/00; A61B 5/024; A61B 5/0261; A61B 5/14542; A61B 5/28; A61B 2562/0204; A61B 2562/0219

(57) ABSTRACT

Systems and methods for promoting sleep in view of the hormonal status of a user are disclosed. Sleep related data for a user is received from one or more sensors. One or more inputs relating to the user's hormonal status are received. One or more recommendations and/or insights are determined to help improve the user's sleep. The one or more recommendations and/or insights are presented to the user.

### CROSS-REFERENCE TO RELATED APPLICATIONS

This application claims the benefit of Australian Patent Application No. 2021904232 filed 23 Dec. 2021, and Australian Patent Application No. 2022901994 filed 15 Jul. 2022, the contents of which are incorporated herein by reference in its entirety.

### 2 BACKGROUND OF THE TECHNOLOGY

### 2.1 Field of the Technology

The present technology relates to systems and methods for monitoring and managing sleep, and may concern systems and methods for assisting a user to improve sleep health.

### 2.2 Background of the Technology

Poor sleep is a significant issue globally. Insufficient rest can have potential negative impact on quality of life, mental health, workplace productivity, health-care utilization, and disease morbidity.

It is known that sleep disturbances for women are pronounced during periods of significant menstrual cycle changes and hormonal fluctuations-such as experienced during pregnancy, menopause, and/or conditions such as polycystic ovary syndrome (PCOS).

However, while a variety of sleep monitoring and sleep improvement products have been (or are) on the market-including wearable devices such as wristwatches, armbands, head mounted devices, and non-contact products—there remains a need for solutions accounting for the particular needs of women.

### 3 BRIEF SUMMARY OF THE TECHNOLOGY

Aspects of the present technology may help with promoting sleep. In particular, aspects of the present technology relate to promoting sleep in view of the hormonal status of a user. For example, a user's hormonal status may be influenced by one or more of: the user's menstrual cycle, pregnancy, menopause, and/or conditions such as polycystic ovary syndrome (PCOS).

Some of the included features may include one or more of: recording sleep related data for a user from one or more sensors; recording inputs relating to one or more of the user's hormonal status, exercise, diet, medication, and/or supplements; offering personalized recommendations and/or insights to help improve the user's sleep; presenting information on the user's hormonal status along with sleep data on a user interface.

According to an aspect of the present technology there is provided a method including: receiving sleep related data for a user from one or more sensors; receiving one or more inputs relating to the user's hormonal status; determining one or more recommendations and/or insights to help improve the user's sleep; and presenting the one or more recommendations and/or insights to the user.

In examples, the one or more sensors may configured to monitor one or more characteristics of a user from which one or more sleep events may be determined. In examples, the one or more sensors may include one or more of: heart rate sensors, oxygen level sensors, ECG sensors, pulse rate sensors, accelerometers, gyroscopes, GPS receivers, sound sensor and/or microphone, and non-contact sensing device. In examples the one or more sensors may include an optical sensor of a wearable sensor device configured to measure a blood volume pulse of the user.

In examples the one or more inputs relating to the user's hormonal status may be user inputs. In examples the one or more user inputs relating to the user's hormonal status may include: Level of tiredness/fatigue (e.g. less, normal, more); Instances of localised pain (e.g. headache, stomach pain, breast pain, back pain); Period characteristics such as: level of bleeding flow (e.g. mild, normal, heavy), pain level (e.g. low, medium, high), associated symptoms (e.g. fatigue, localised pain, cramping, mood); Period timing characteristics of current or previous cycle (e.g. start, end, duration, regularity, missed periods); Medication impacting menstrual cycle (e.g. to delay onset of period); and/or Other factors such as instances of hot flashes, nausea, constipation, acne, mood, and/or appetite.

In examples, the one or more user inputs may be received in response to a questionnaire. In examples the one or more user inputs may be received via a questionnaire interface presented to the user. In examples the one or more user inputs may be entered by another party conducting a sleep assessment for the user.

In examples, the user may be provided with means for entering diary notes—for example an open field or free-form text.

In examples, one or more of the more inputs relating to the user's hormonal status may be determined using one or more characteristics of the user obtained using one or more sensors. In examples, one or more of the more inputs relating to the user's hormonal status may be obtained through biological testing. For example, one or more of the more inputs relating to the user's hormonal status obtained through biological testing may comprise one or more of: glucagon, epinephrine (adrenaline), norepinephrine, cortisol, and growth hormone.

In examples, one or more inputs relating to the user's exercise habits may be received. In examples the one or more inputs relating to the user's exercise habits may be received in response to the questionnaire. In examples the one or more inputs relating to the user's exercise habits may be received from one or more exercise applications (e.g. Apple™ Fitness+, Fitbit™, MyFitnessPal™, or Samsung™ Health).

Generally, recommendations will focus on helping the user plan and prepare factors such as their sleep environment, nightly habits/routine, physical readiness, emotions, beliefs, and mindset for the anticipated sleep disturbances to happen based on their hormonal status and/or other predictable causes (i.e. daily dairy entry, comorbidities, past sleep data, etc.). Recommendations may also provide general guidance to help improve sleep and achieve the user's desired sleep goals through general best practices, sleep education, and known science.

In examples, the one or more recommendations may include: Lifestyle changes; Sleep hygiene; Auto Sleep Diary; Ideal bed time; Sleep programs; Relaxation exercises; Educational articles; Consulting a sleep consultant; Consulting a medical professional; and/or Screening for a sleep related disorder.

In examples, the one or more insights may include: Ovulation period; Hormone fluctuations; Effect of hormone fluctuations on sleep; Correlation between a menstrual cycle phase (e.g. menstrual, follicular, ovulation, luteal phases) and an indicator of sleep of the user; Correlation between an indicator of sleep and user entered factors; An indicator of readiness (i.e. how mentally and/or physically ready the user is to take on the day); and/or Guidance on factors which appear to be impacting the user's sleep and/or readiness.

In examples, dietary recommendations may be provided in response to user inputs indicative of diet affecting the user's sleep. For example, one or more user inputs in response to the questionnaire may include key words indicative of dietary factors impacting sleep.

In examples, a recommendation to consult with a specialist may be provided in response to user inputs indicative of medication and/or supplements affecting the user's sleep. For example, one or more user inputs in response to the questionnaire may include key words indicative of medication and/or supplements impacting sleep.

In examples, insights regarding the impact of hormonal status on sleep may be provided in response to sensor and/or user inputs indicative of hormonal status affecting the user's sleep.

In examples, recommendations for relieving stress and/or insights regarding the impact of stress on sleep may be provided in response to user inputs indicative of stress affecting the user's sleep.

In examples, a prediction of the user suffering from problems with falling or staying asleep may be made based on monitoring of one or more factors over an extended period of time. In examples the extended period of time may be at least seven days. In examples the extended period of time may be at least fourteen days. In examples the factors may include one or more of: caffeine intake, sleep start time, diet, supplements, medications, symptoms (e.g. headaches, uterine cramps, etc), menstrual cycle phase. In examples the factors may include one or more of: average cycle length (e.g. <21 days, 21-35 days, >35 days, no cycles), contraceptive use (e.g. pills, patches, hormonal IUD, implant, intravaginal ring, injection, etc), pregnancy, lactation, Polycystic Ovarian Syndrome (PCOS), menstrual related disorders (e.g. heavy menstrual bleeding, no menstrual bleeding, premenstrual syndrome, premenstrual dysphonic disorder), perimenopause, menopause. In examples the prediction of the user suffering from problems with falling or staying asleep may be based in part on monitoring of other factors such as improving sleep habits, life experiences (for example, quality of life, or perceived status of relationships), and stress relief activity.

According to an aspect of the present technology there is provided a method including: receiving sleep related data for a user from one or more sensors; receiving one or more inputs relating to the user's hormonal status; and presenting information on the user's hormonal status along with sleep data on a user interface.

According to an aspect of the present technology there is provided a virtual sleep clinic comprising one or more of: sleep assessment services; on-demand telehealth visits with a licensed practitioner who is attuned to female sleep issues and who can diagnose sleep disorders, provide behavioural therapy, and/or prescribe therapy; and provision of sleep solutions designed for female sleep needs.

In examples a screening stage may be performed to determine a sleep assessment pathway for a user. In examples the screening stage may identify whether the user is likely to be impacted by male or female hormonal status, and direct the user to a sleep pathway based at least in part on this identification. In examples a first sleep assessment pathway may be directed to sleep respiratory conditions (for example sleep disordered breathing such as Obstructive Sleep Apnea). In examples a second sleep assessment pathway may be directed to problems with falling or staying asleep.

According to an aspect of the present technology there is provided a system comprising one or more processors, and at least one memory having stored thereon machine-readable instructions, wherein the processors are configured to execute the machine-readable instructions to perform a method as described herein.

In examples the system may comprise an electronic interface configured to receive user input and/or to provide user instructions. In examples the system may comprise a head-mounted display device—for example an augmented reality head-mounted display, or a virtual reality head-mounted display—and at least a component of the electronic interface is provided via the head-mounted display device. In examples the electronic interface may comprise a virtual clinic environment.

According to an aspect of the present technology there is provided a computer program product comprising instructions which, when executed by a computer, cause the computer to carry out a method as described herein. In examples the computer program product comprises a non-transitory computer readable medium.

The methods, systems, devices and apparatus described may be implemented so as to improve the functionality of a processor, such as a processor of a specific purpose computer, sleep monitor, respiratory monitor and/or a respiratory therapy apparatus. Moreover, the described methods, systems, devices and apparatus can provide improvements in the technological field of automated management, monitoring and/or treatment of sleep related conditions-such as sleep respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

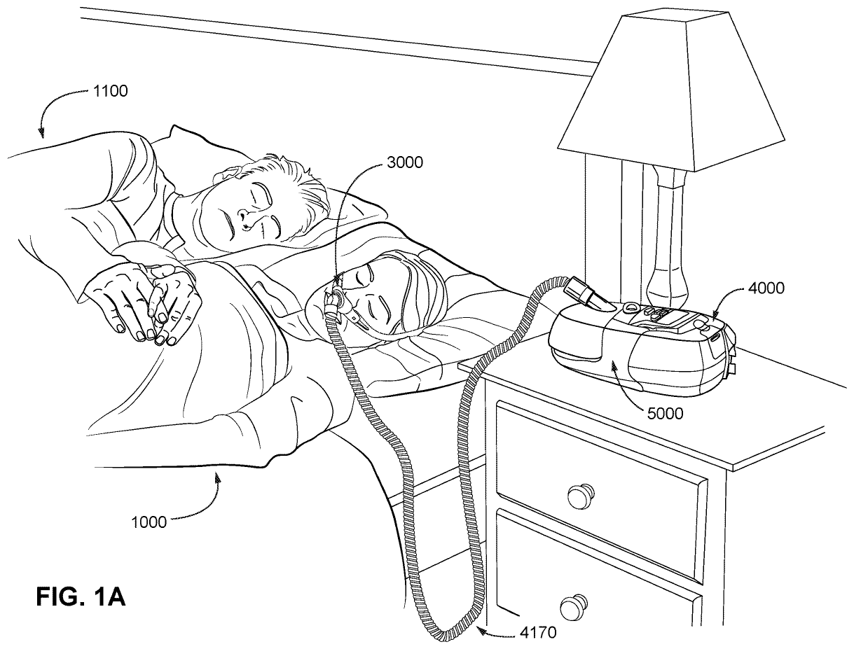

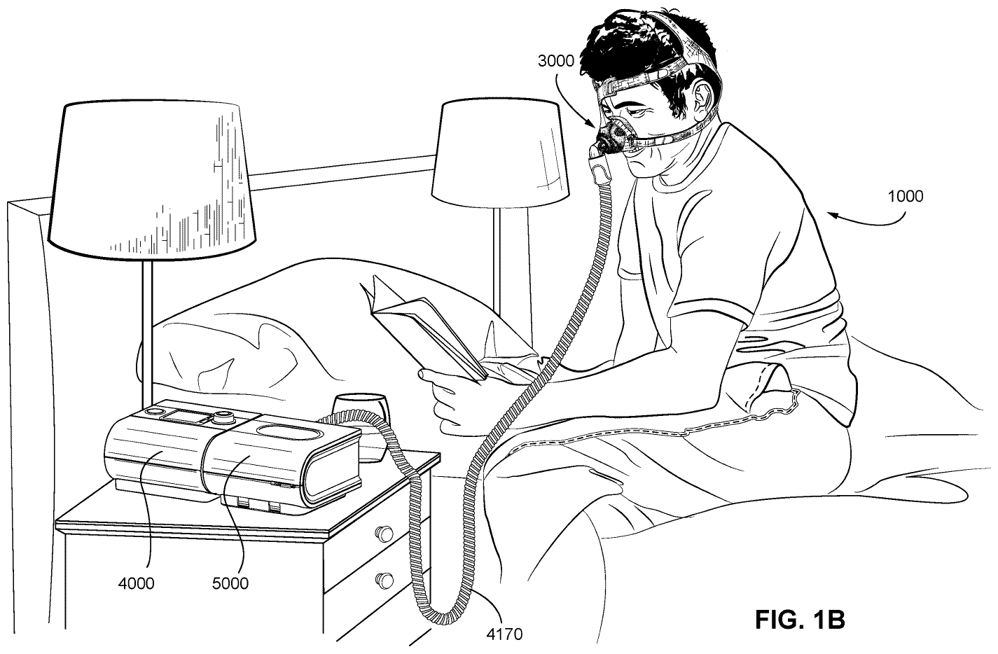

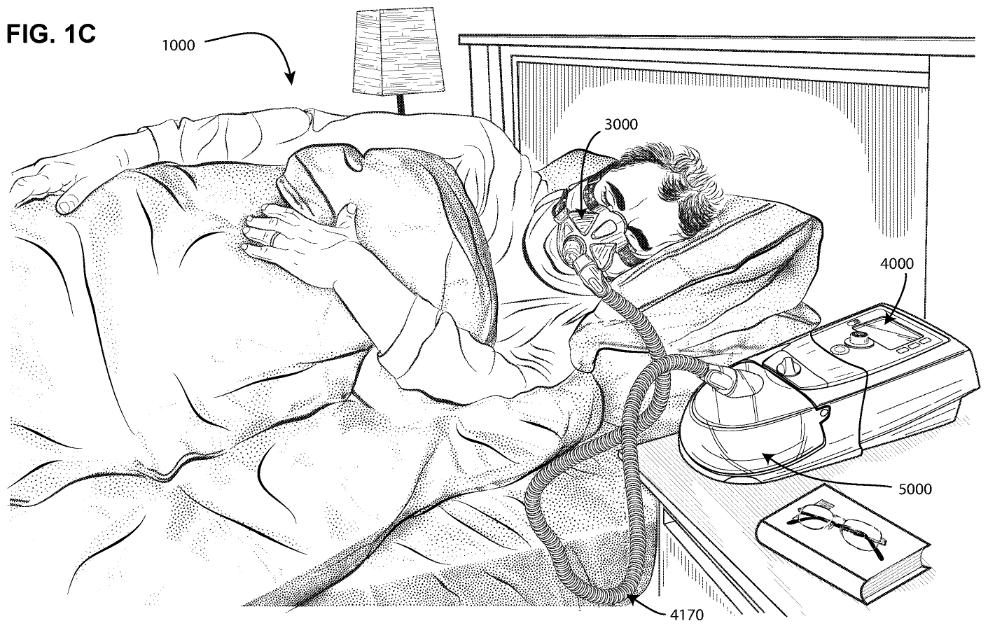

FIG. 1A shows a system including a patient 1000 wearing a patient interface 3000, in the form of nasal pillows, receiving a supply of air at positive pressure from an RPT device 4000. Air from the RPT device 4000 is conditioned in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

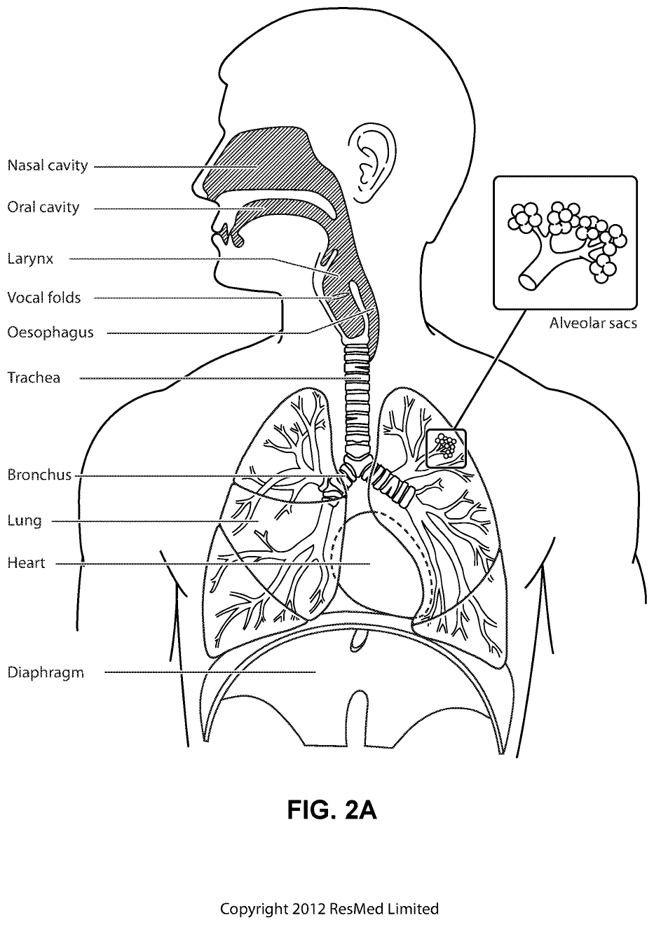

FIG. 2A shows an overview of a human respiratory system including the nasal and oral cavities, the larynx, vocal folds, oesophagus, trachea, bronchus, lung, alveolar sacs, heart and diaphragm.

### 4.3 Patient Interface

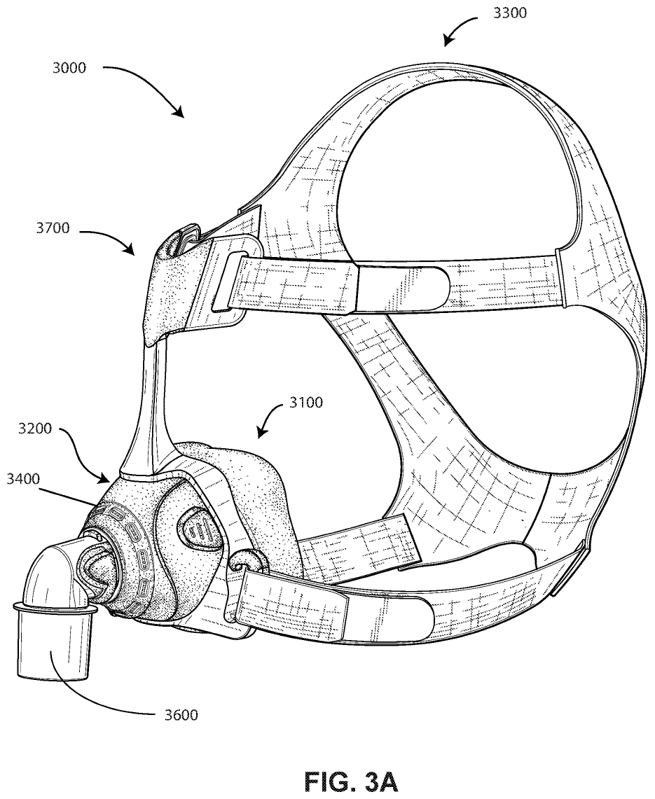

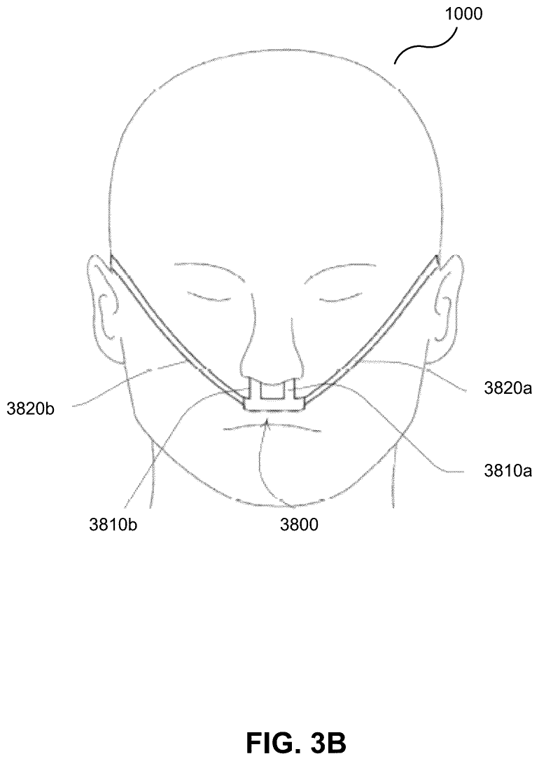

FIG. 3A shows a patient interface in the form of a nasal mask in accordance with one form of the present technology.

FIG. 3B shows a patient interface in the form of a nasal cannula in accordance with one form of the present technology.

### 4.4 RPT Device

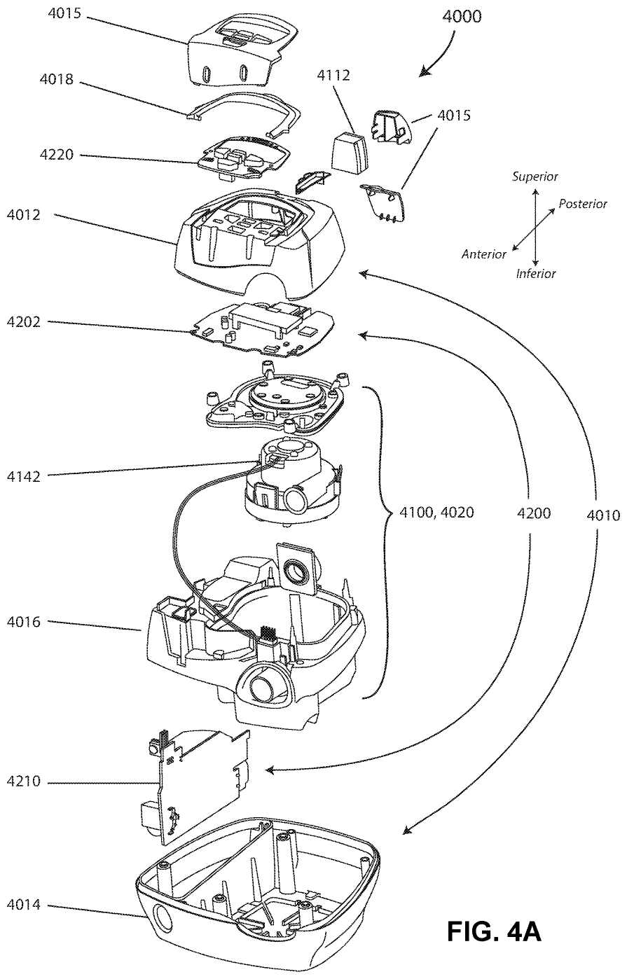

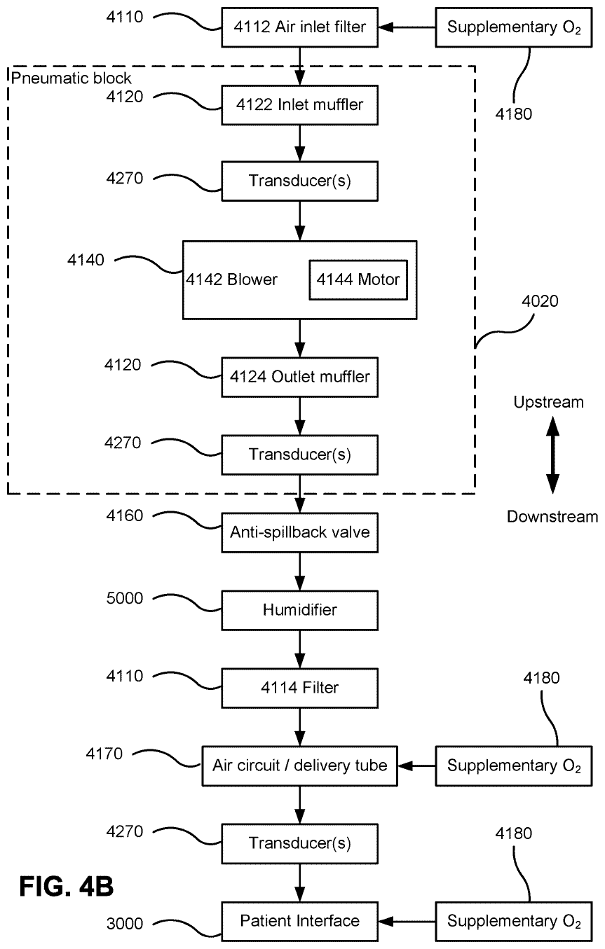

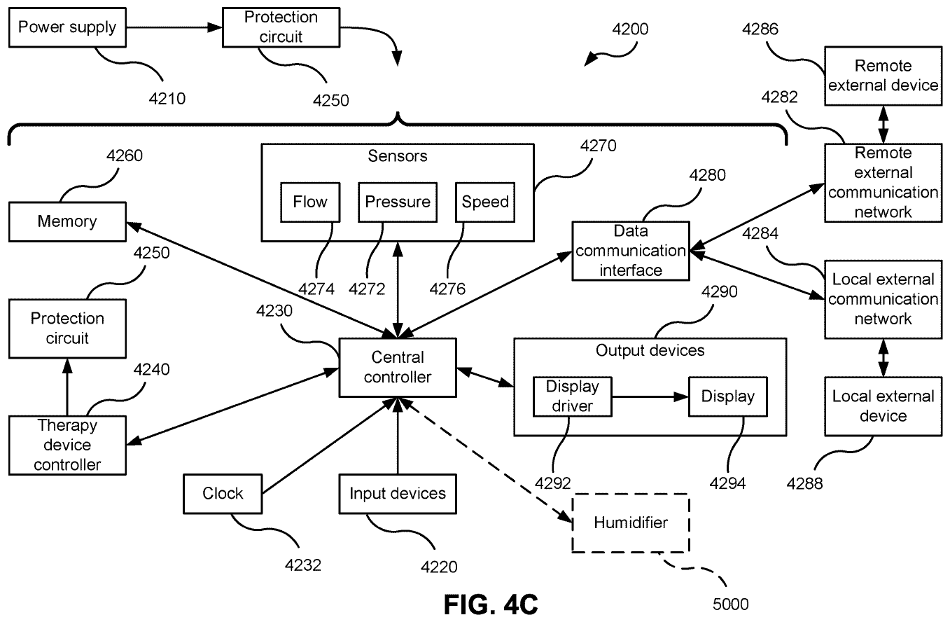

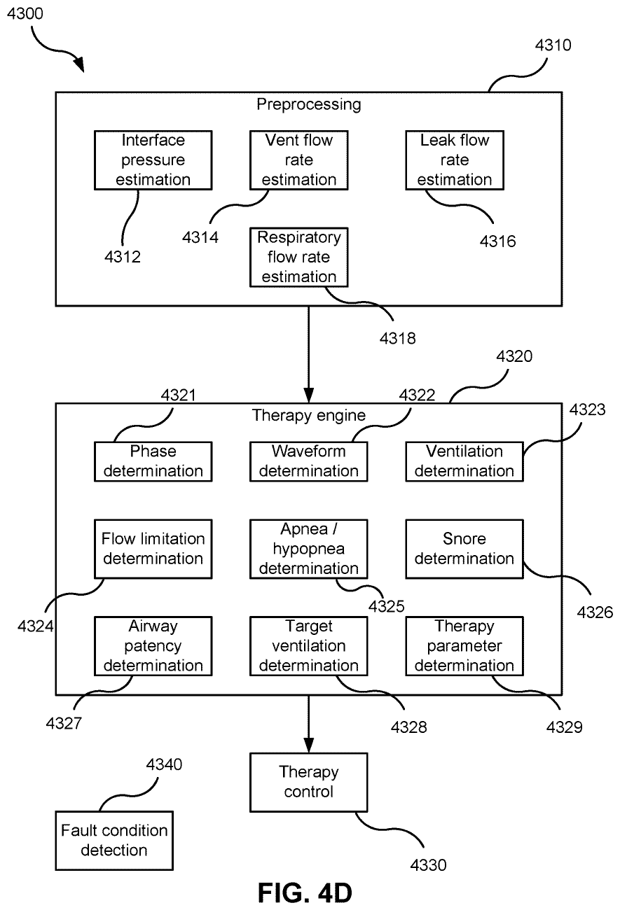

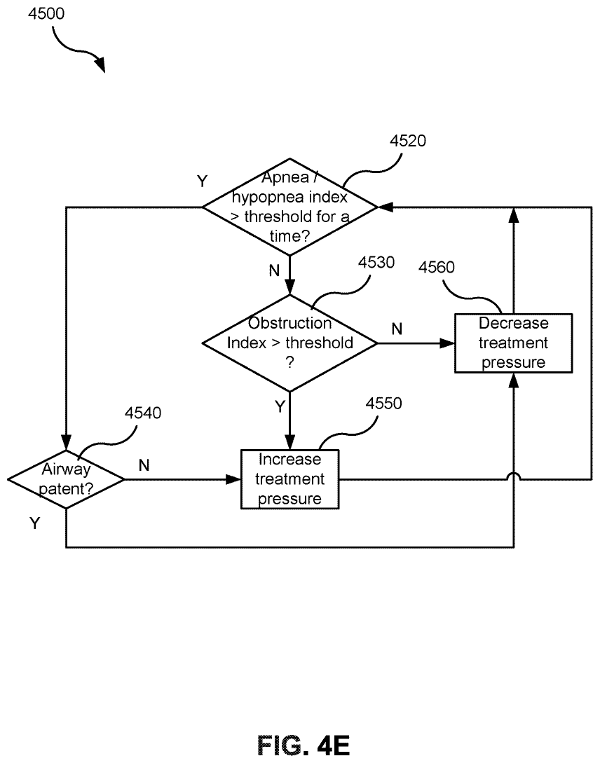

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 4C is a schematic diagram of the electrical components of an RPT device in accordance with one form of the present technology.

FIG. 4D is a schematic diagram of the algorithms implemented in an RPT device in accordance with one form of the present technology.

FIG. 4E is a flow chart illustrating a method carried out by the therapy engine module of FIG. 4D in accordance with one form of the present technology.

4.5 HUMIDIFIER

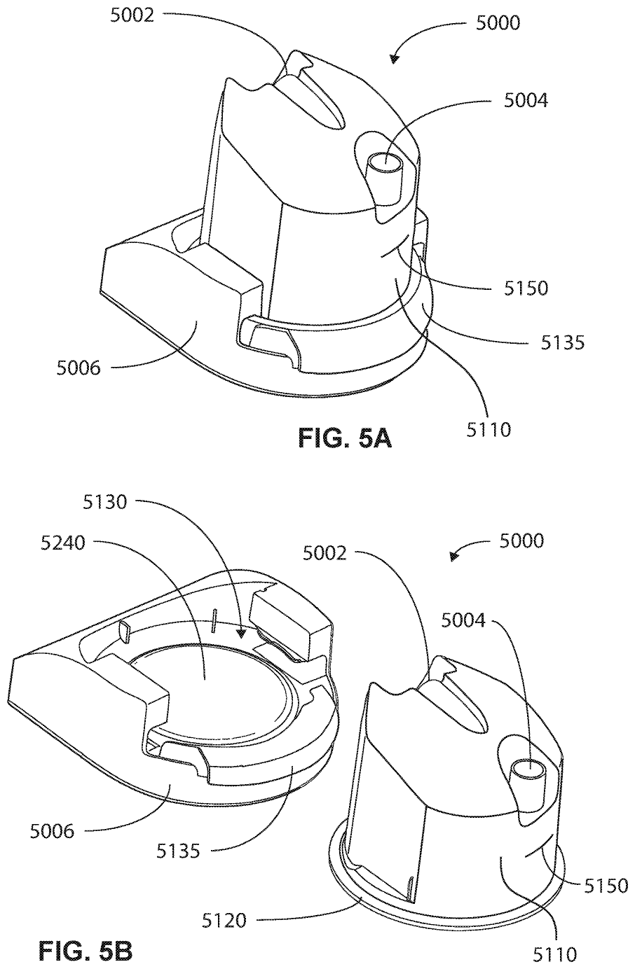

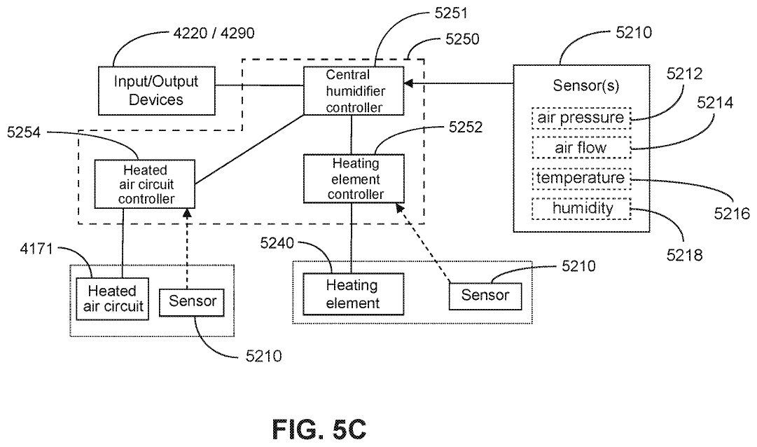

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

FIG. 5C shows a schematic of a humidifier in accordance with one form of the present technology.

### 4.6 Breathing Waveforms

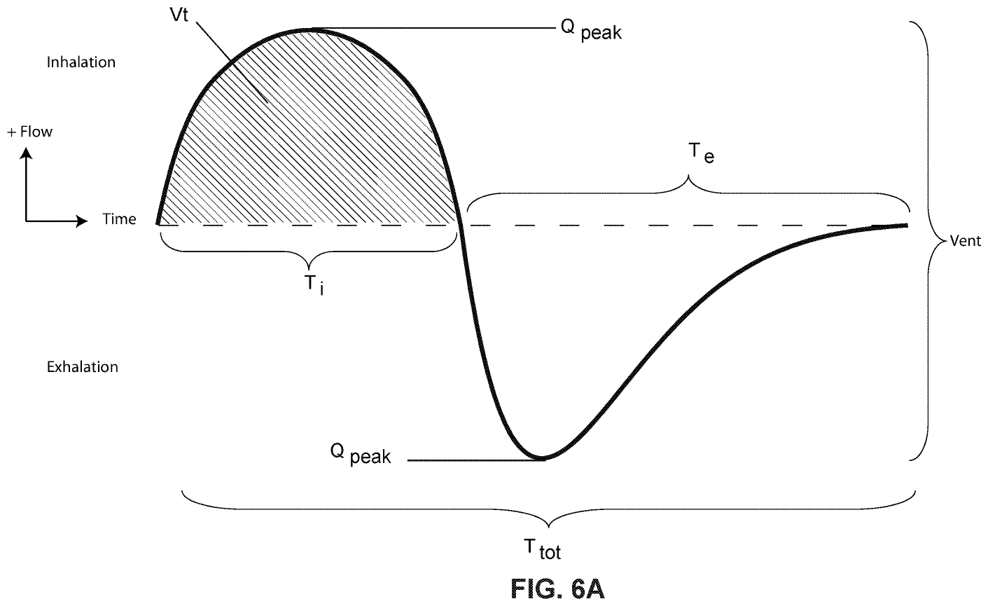

FIG. 6A shows a model typical breath waveform of a person while sleeping.

### 4.7 Screening, Diagnosis and Monitoring Systems

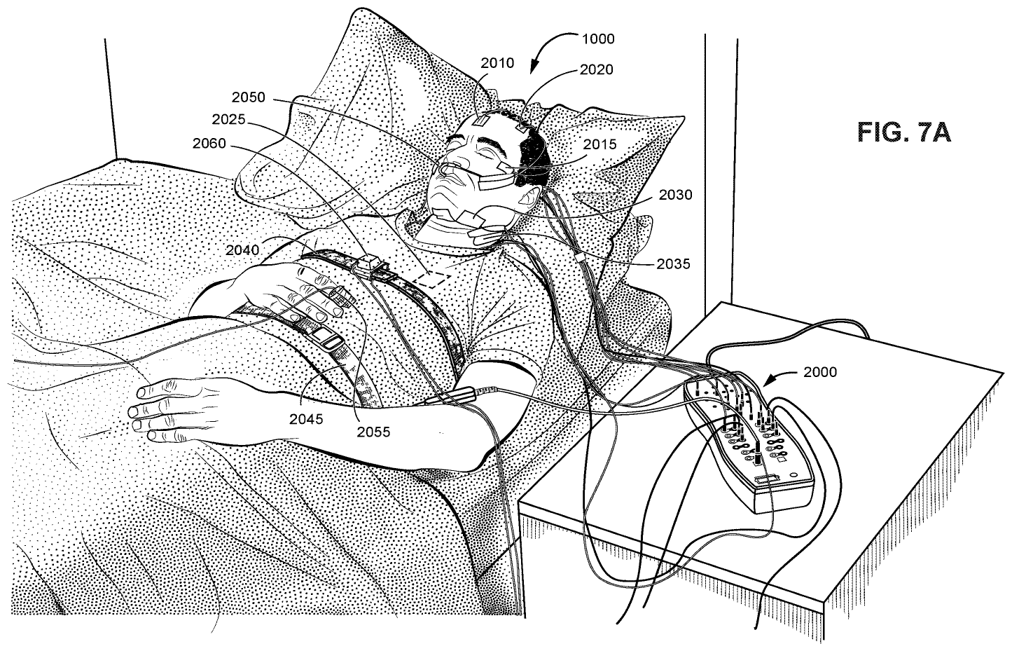

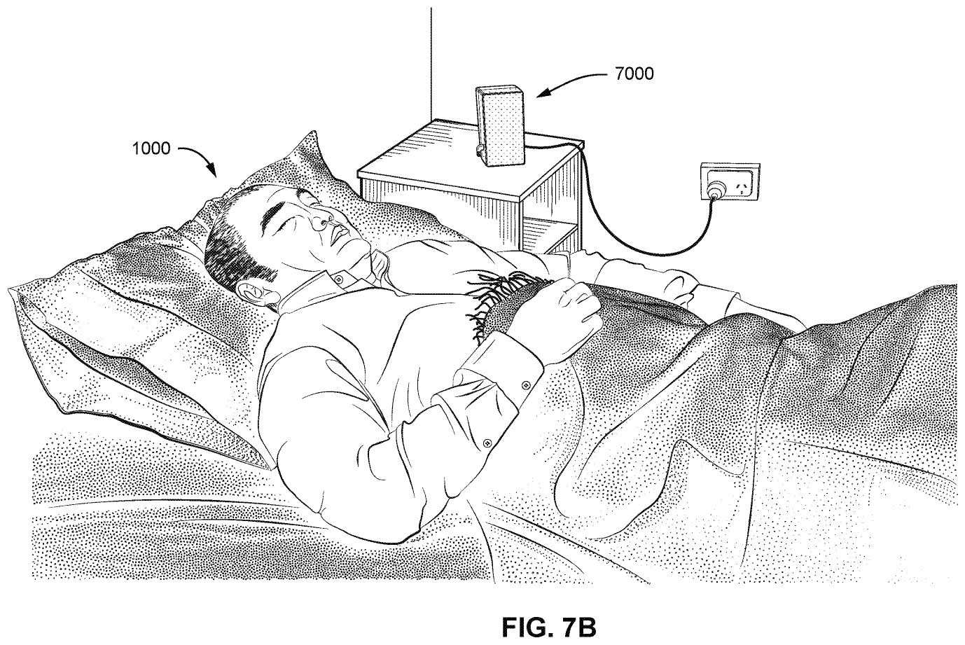

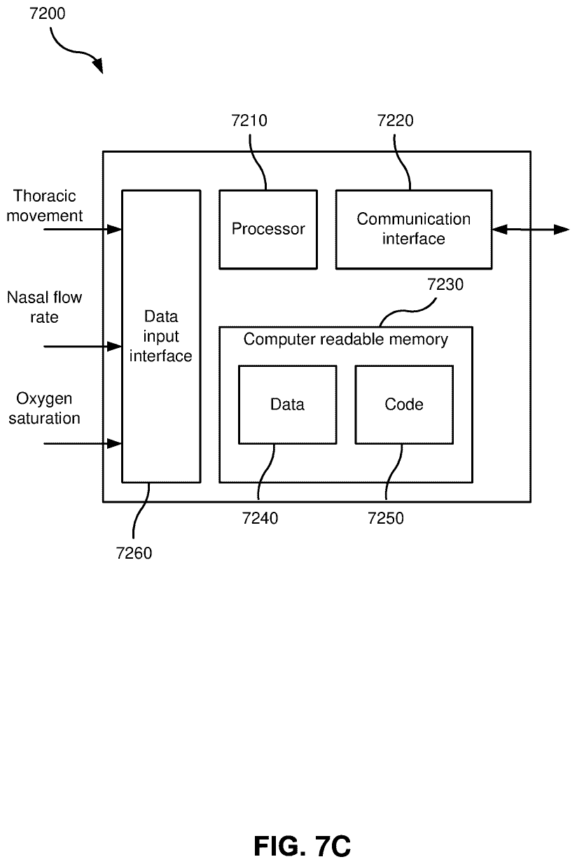

FIG. 7A shows a patient undergoing polysomnography (PSG). The patient is sleeping in a supine sleeping position.

FIG. 7B shows a monitoring apparatus for monitoring the condition of a patient. The patient is sleeping in a supine sleeping position.

FIG. 7C is a schematic diagram of the components of a screening/diagnosis/monitoring device that may be used to implement a Respiratory polygraphy (RPG) headbox in an RPG screening/diagnosis/monitoring system concentrator in accordance with one form of the present technology.

### 4.8 Oxygen Concentrator

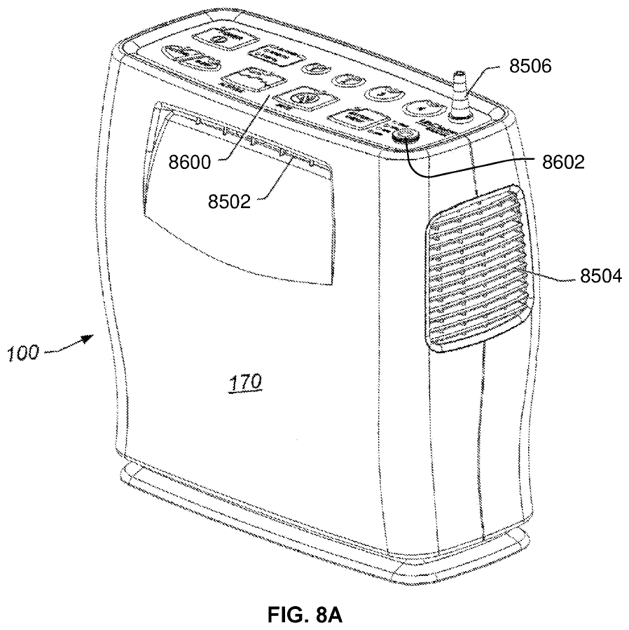

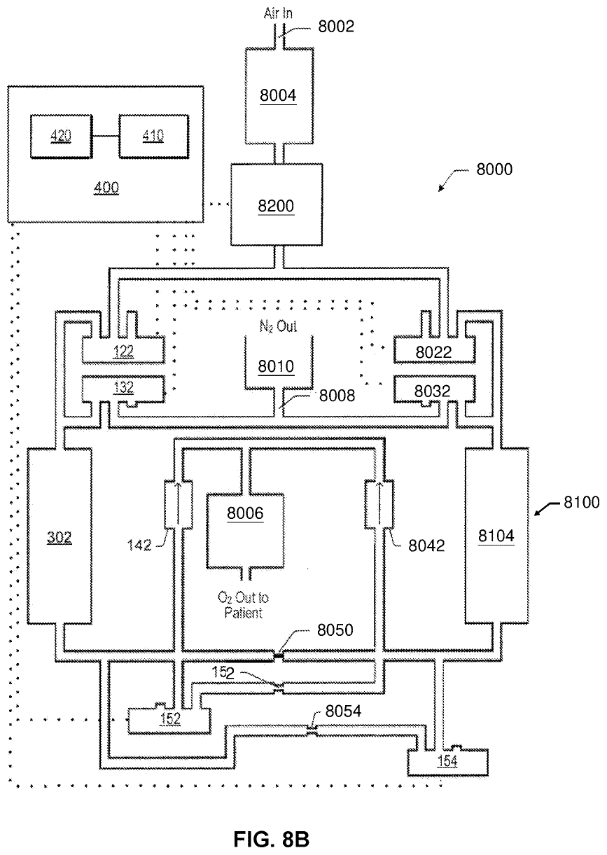

FIG. 8A depicts an oxygen concentrator in accordance with one form of the present technology.

FIG. 8B is a schematic diagram of the components of the oxygen concentrator of FIG. 8A.

### 4.9 Computing System and Processes

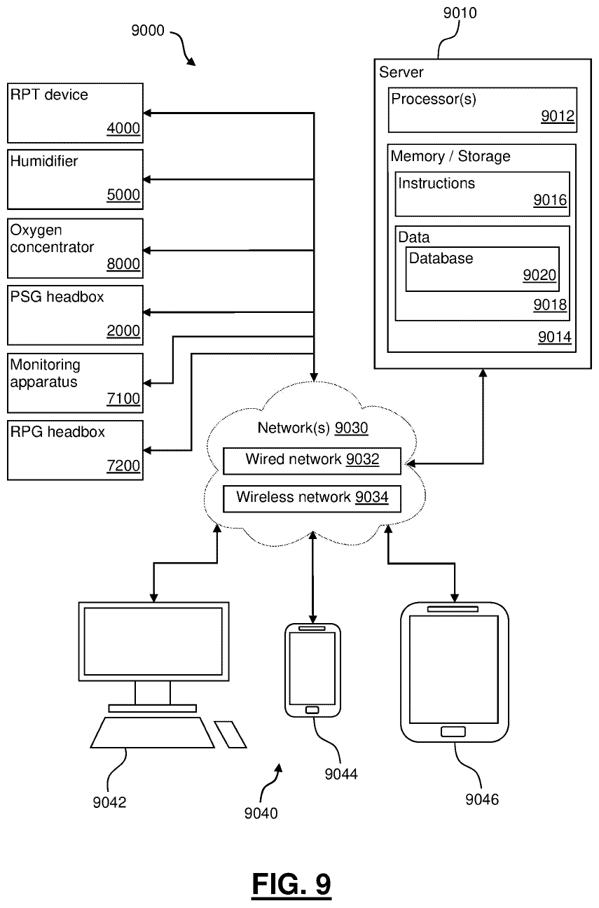

FIG. 9 is a diagram of an example system for monitoring sleep and providing insights and/or recommendations, which includes a computing device;

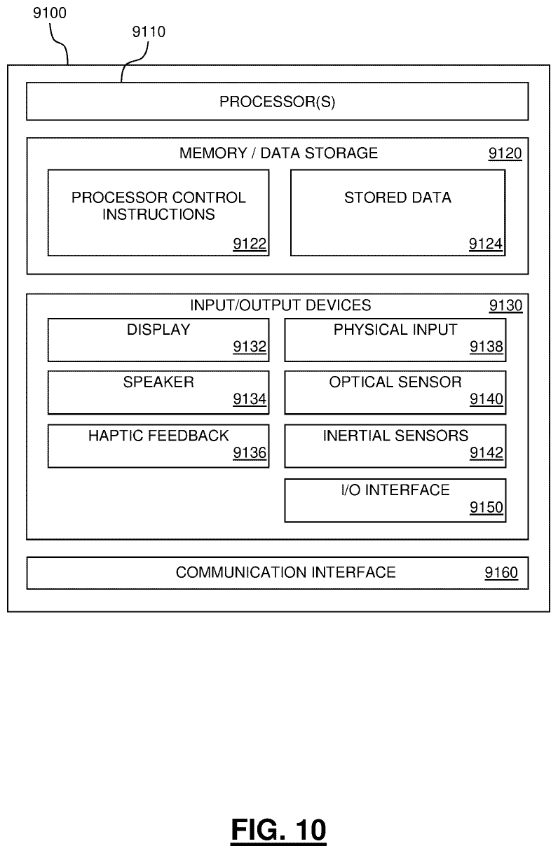

FIG. 10 is a diagram of the components of an example computing device used for monitoring sleep and providing insights and/or recommendations;

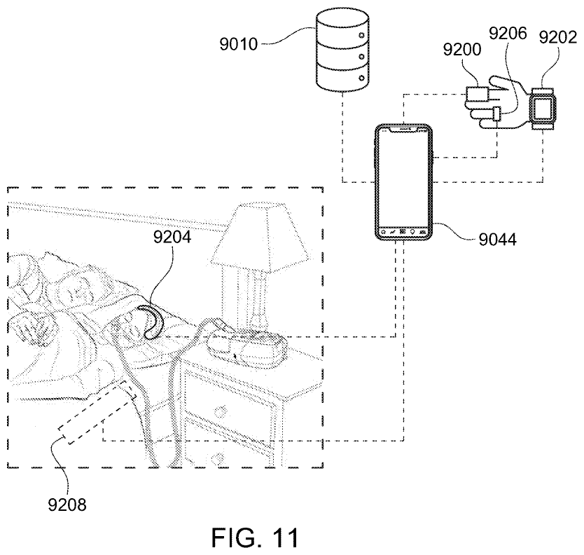

FIG. 11 is a diagram of an example sub-system for collection of sleep related data from a user;

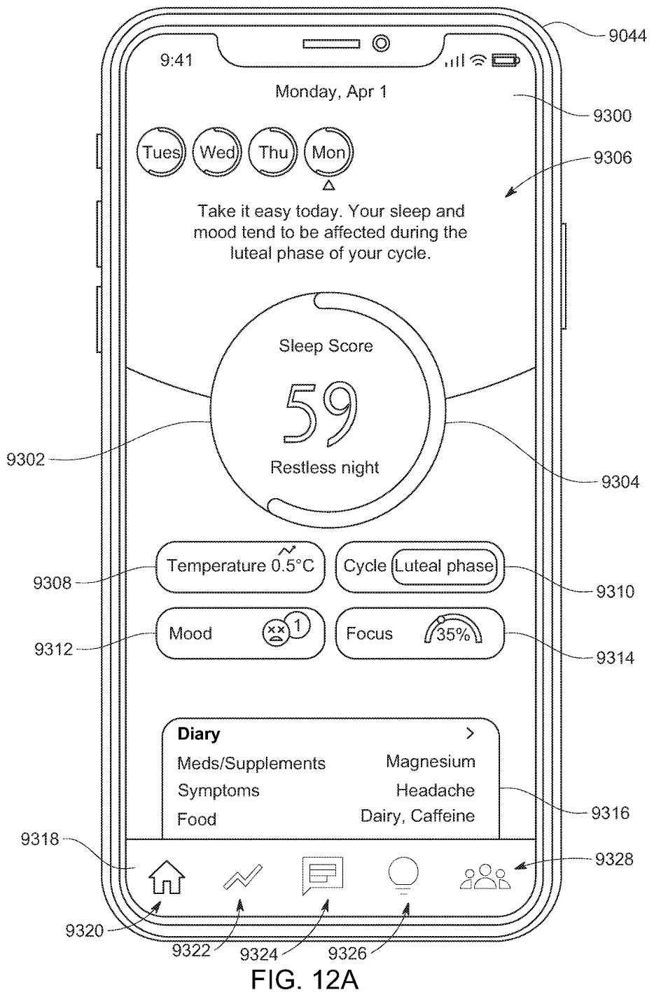

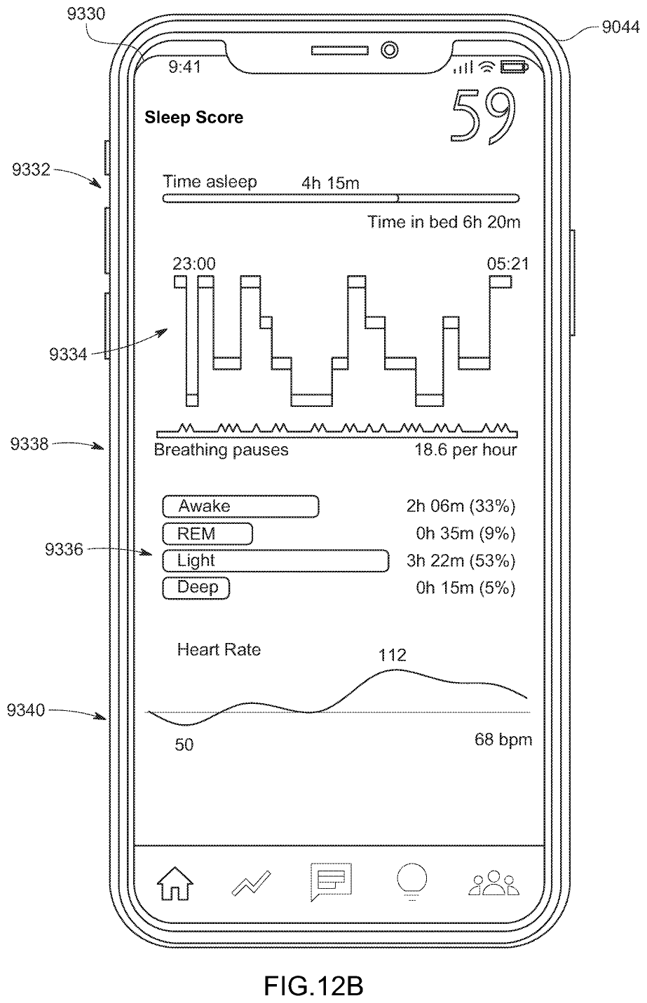

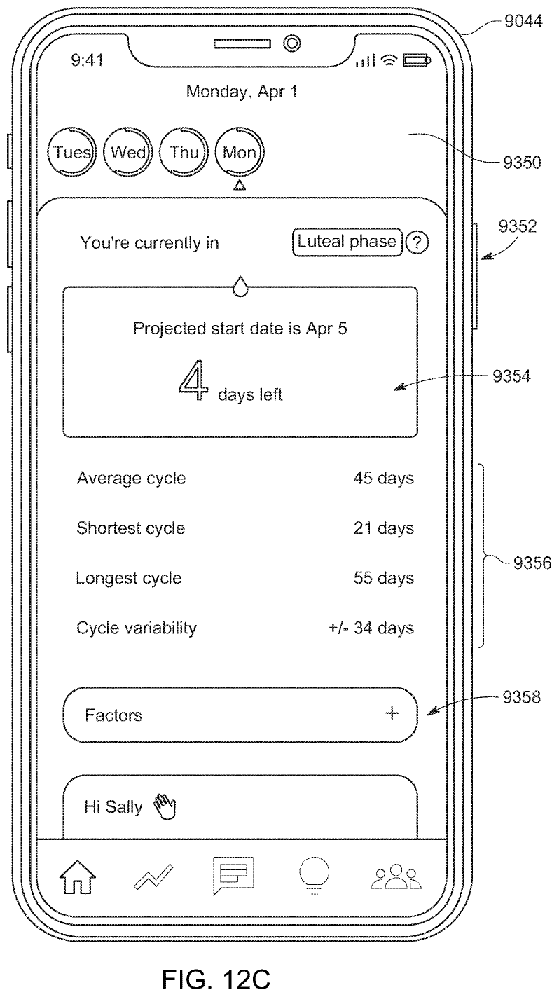

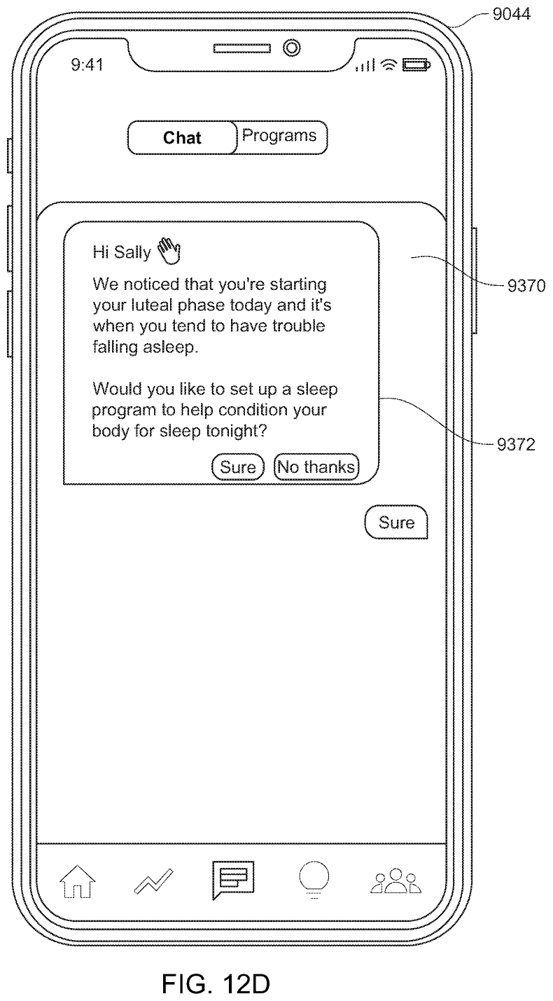

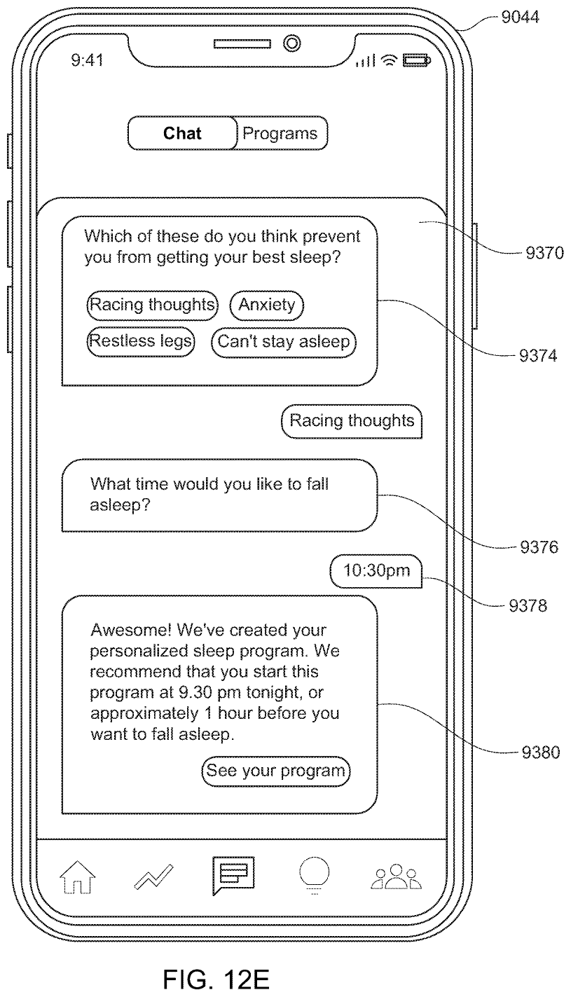

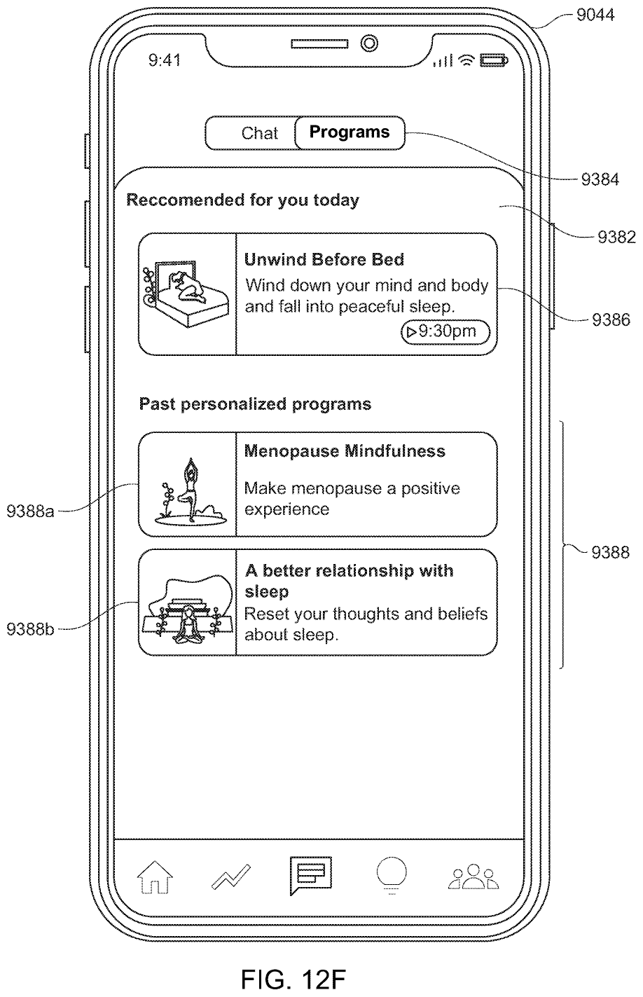

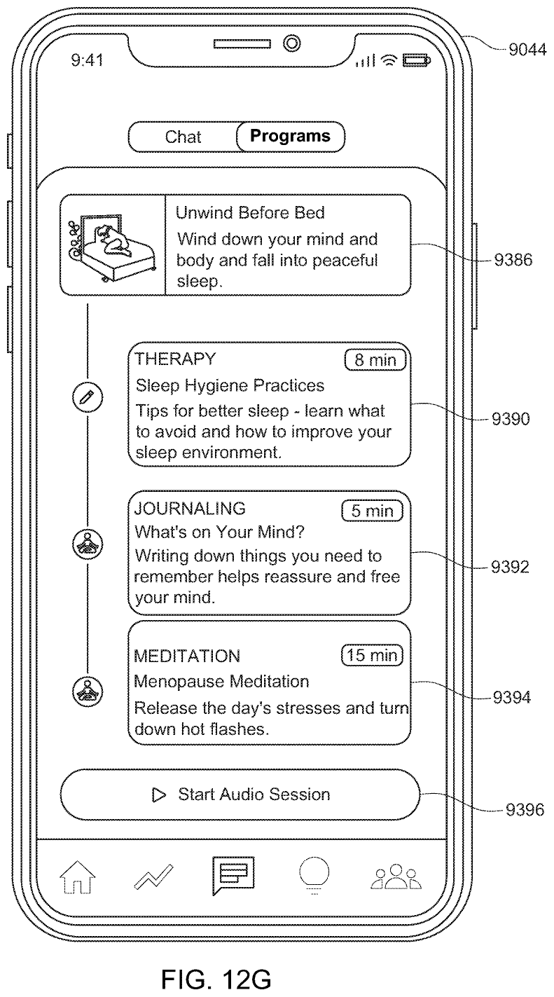

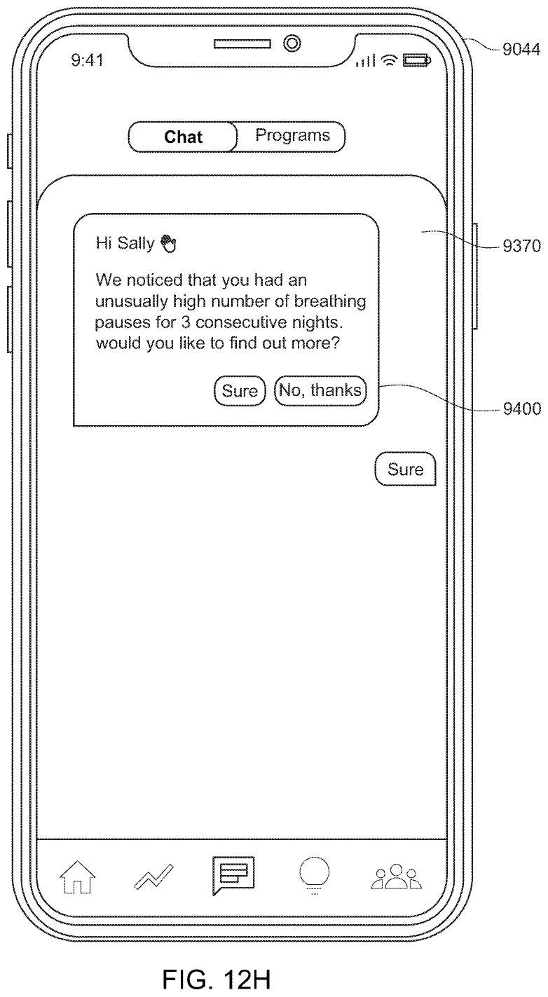

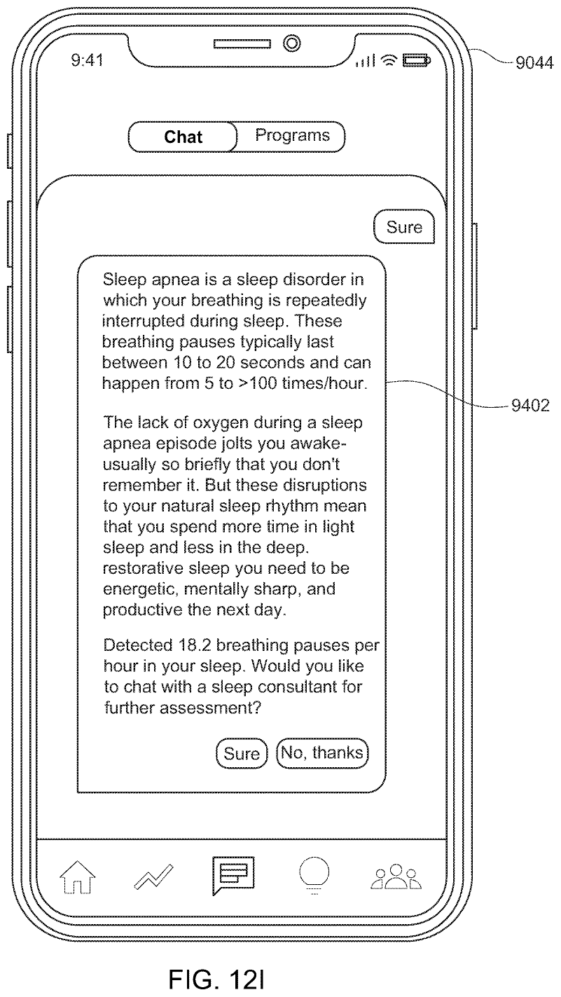

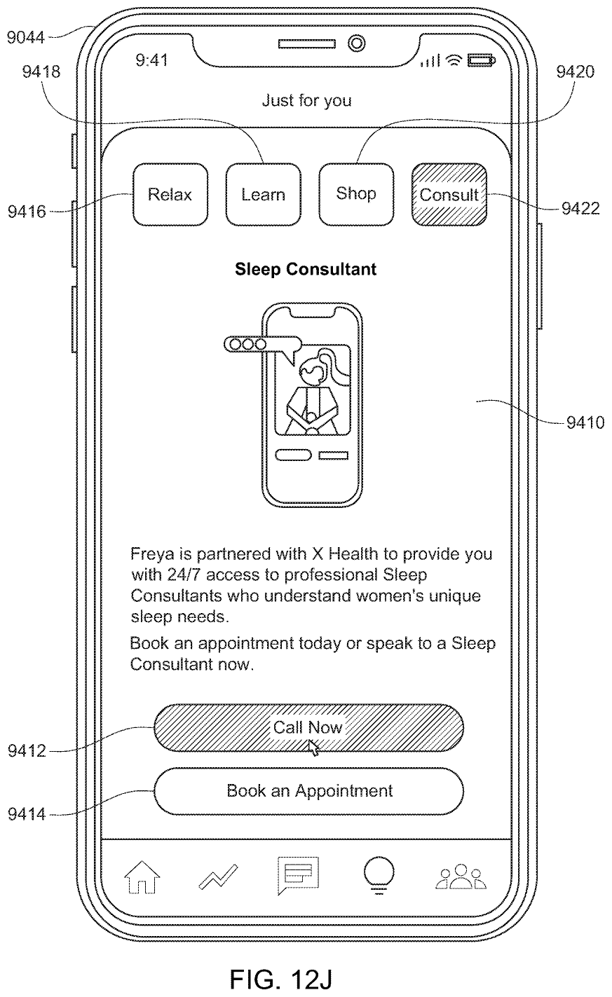

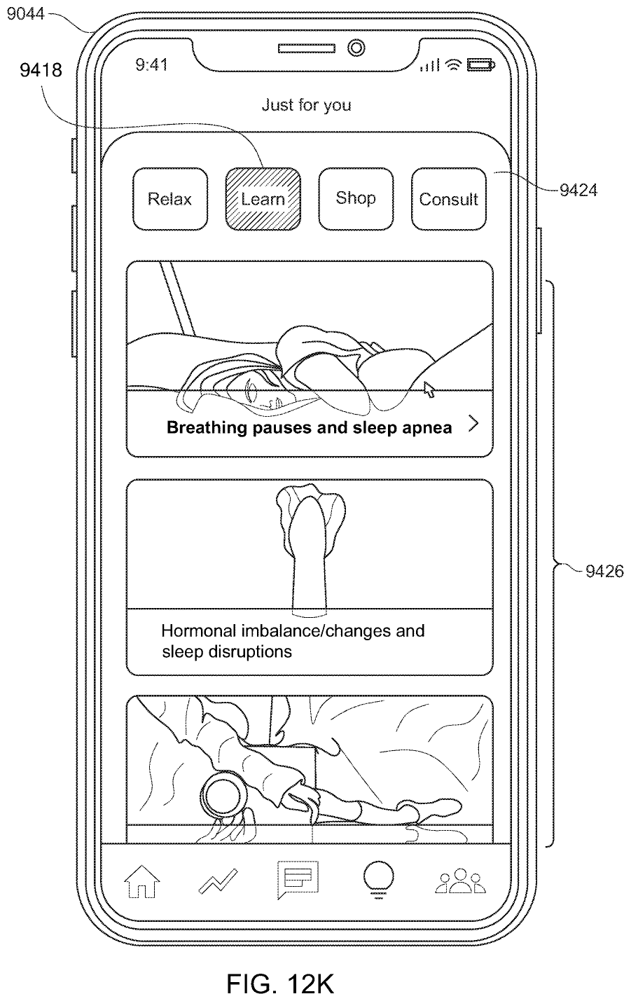

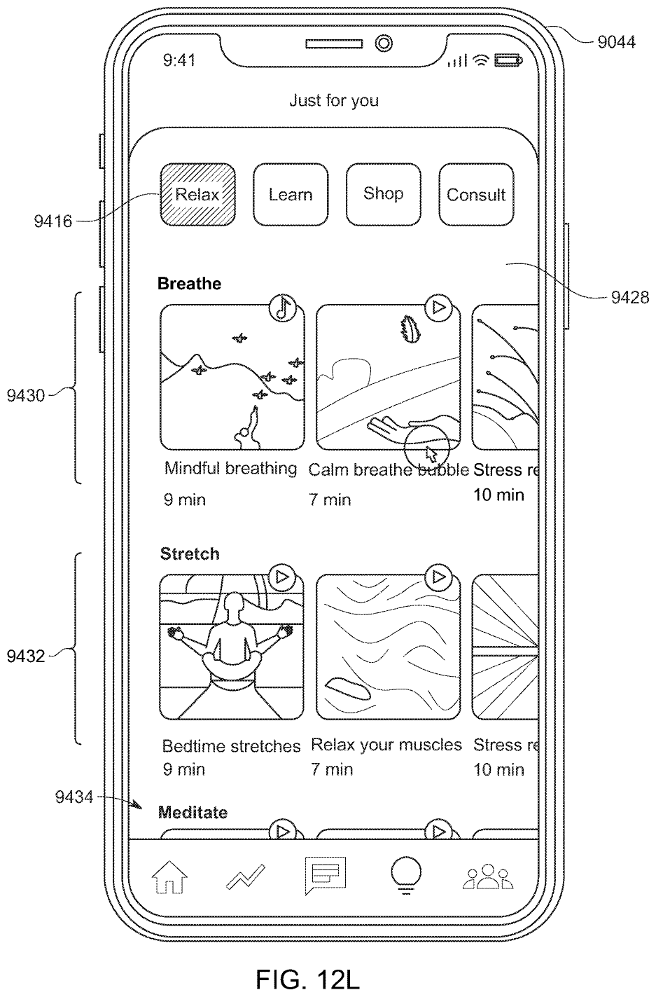

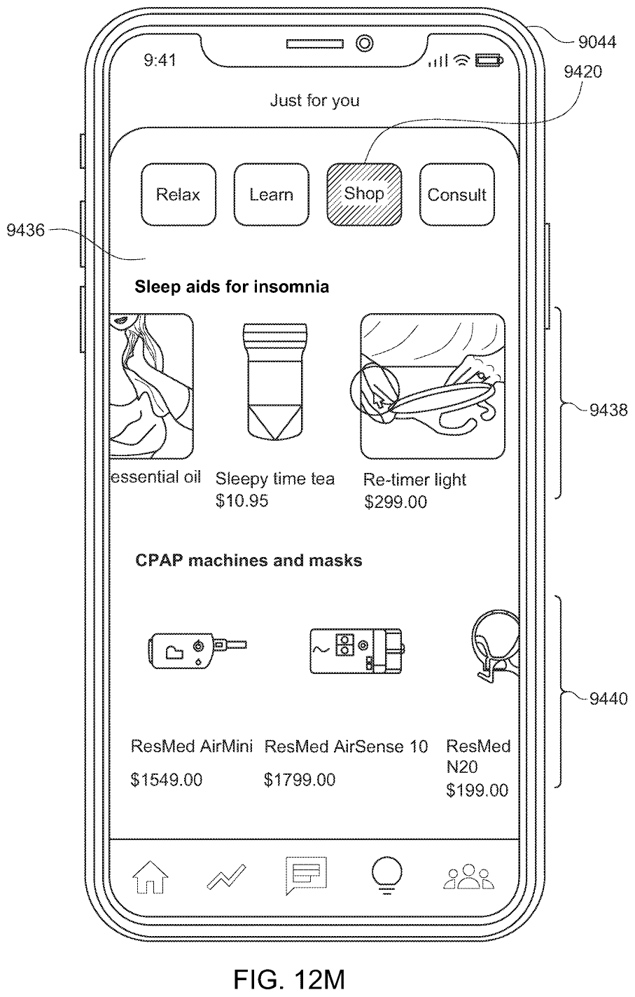

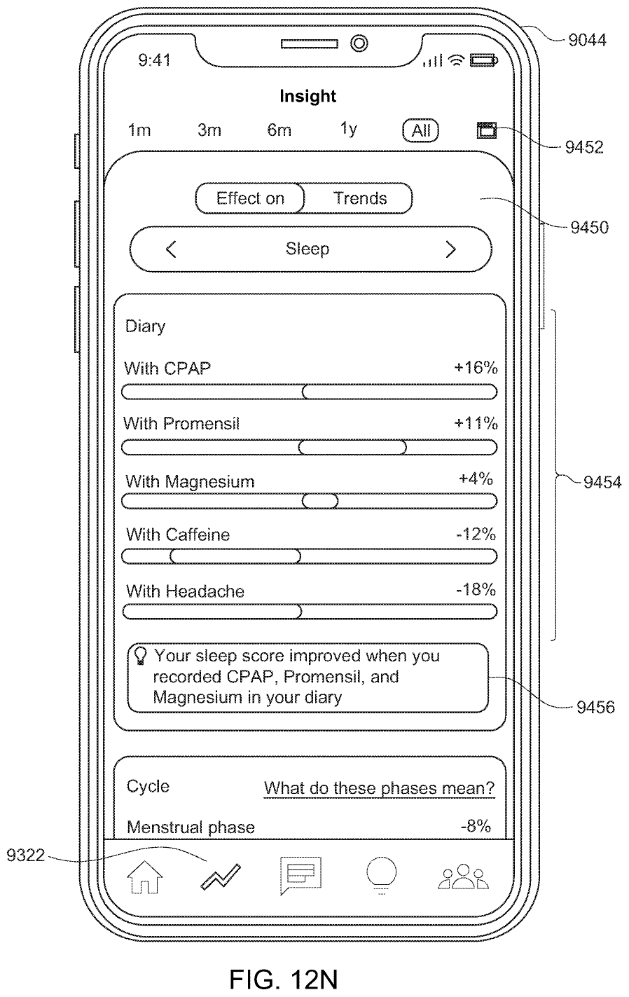

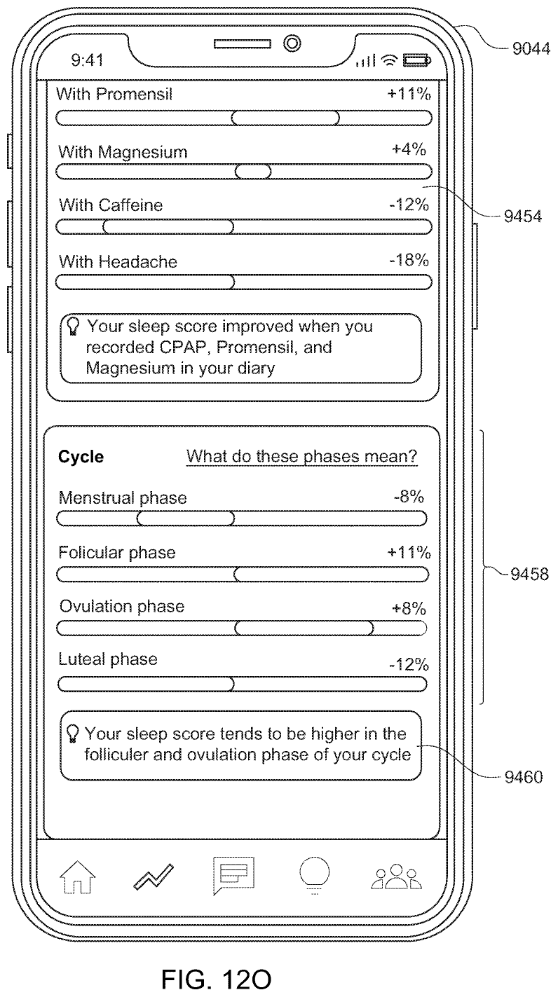

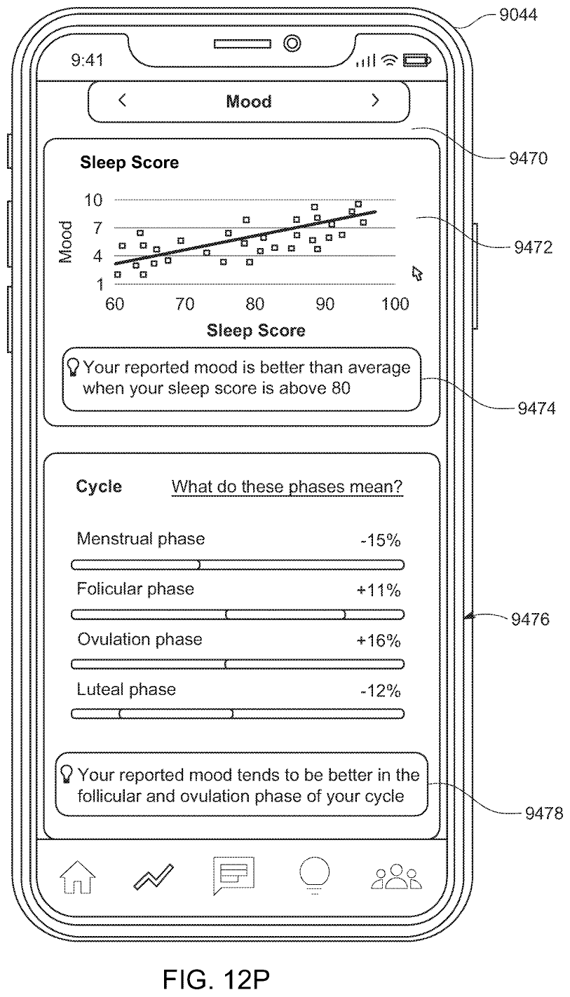

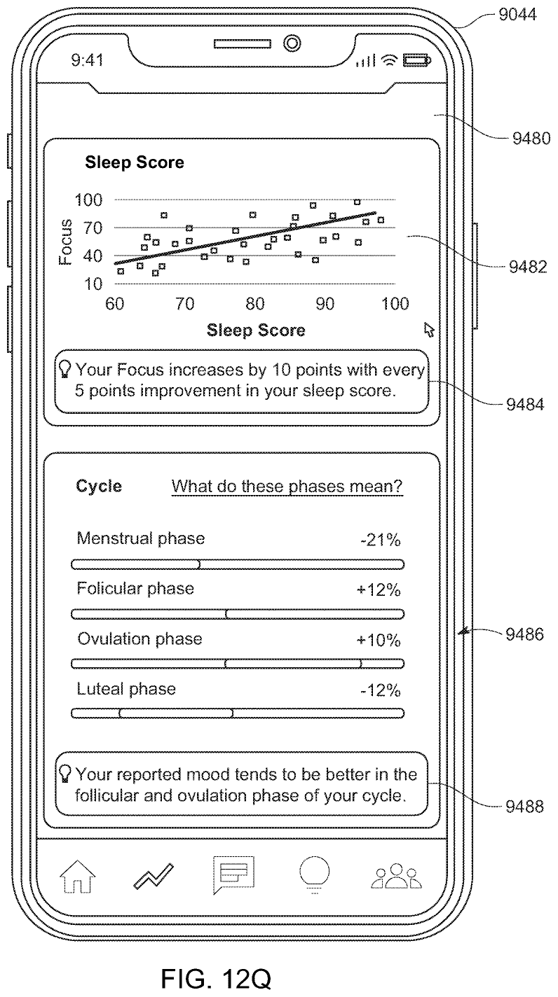

FIGS. 12A-12Q are screen images of interfaces on an example user device for providing sleep related insights and/or recommendations to a user; and

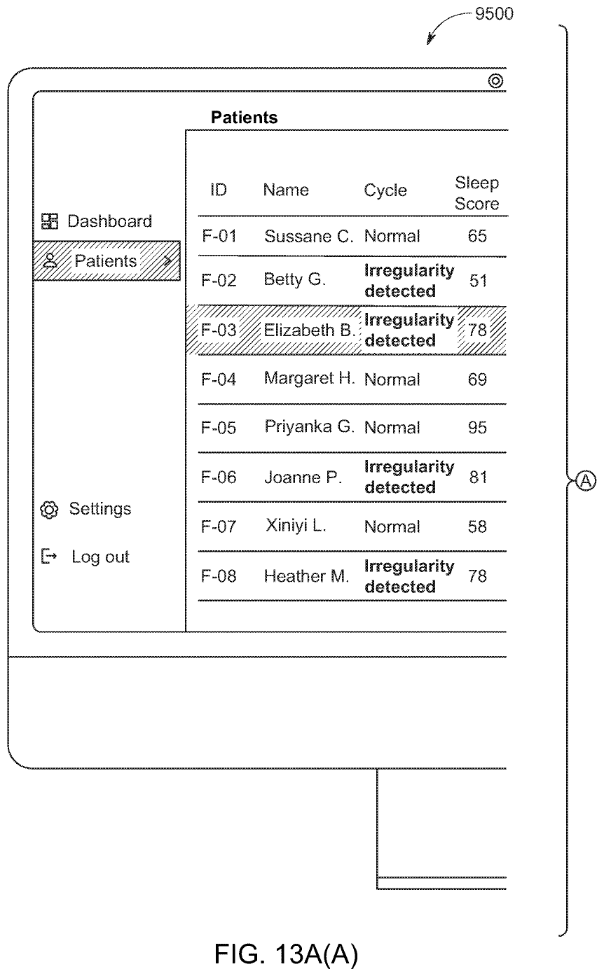

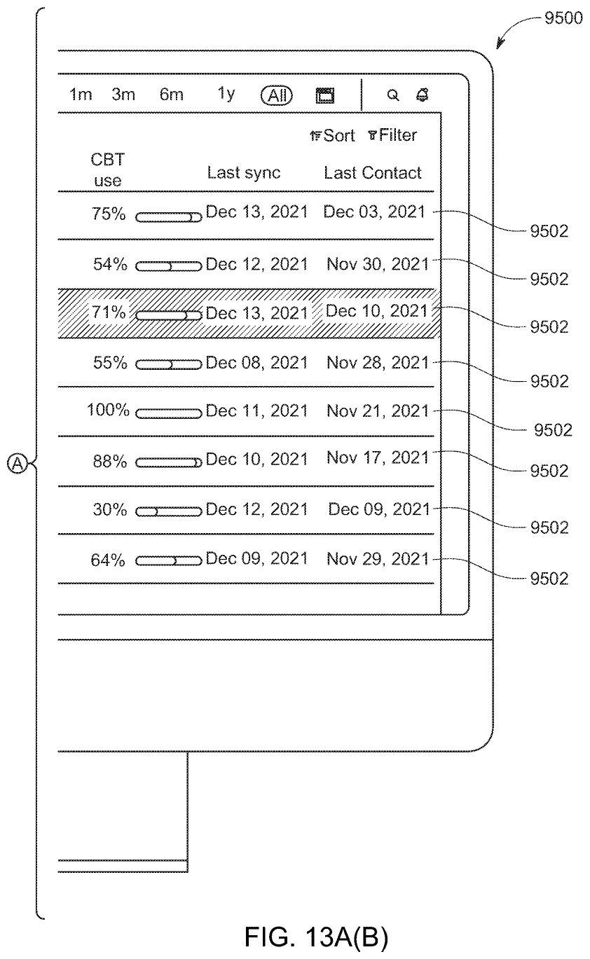

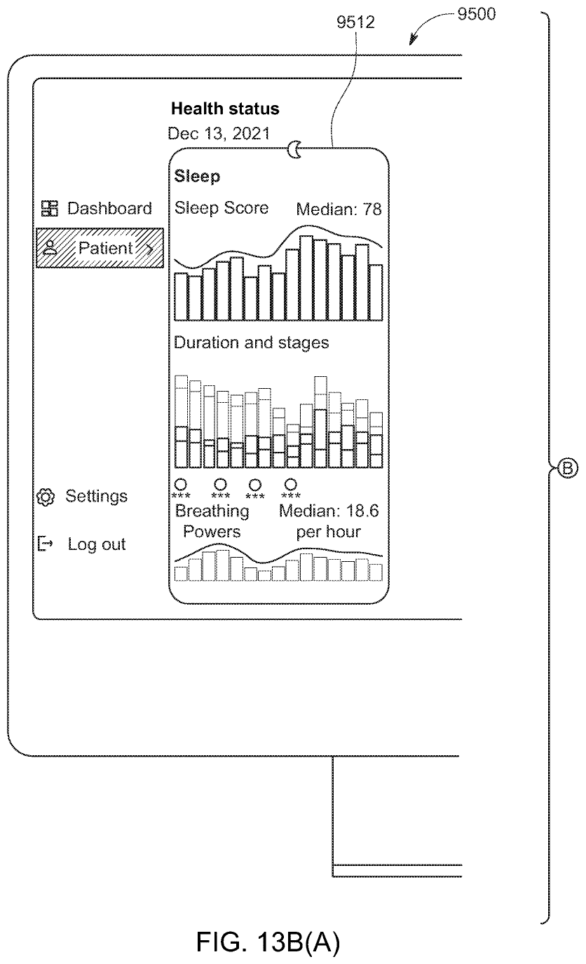

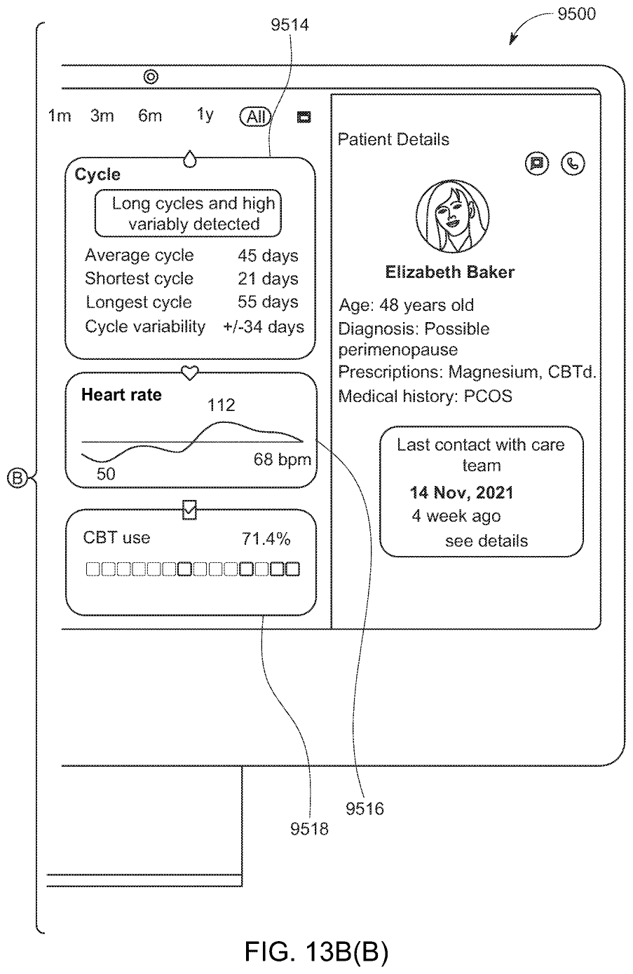

FIGS. 13A(A)/13A(B) and 13B(A)/13B(B) are screen images of interfaces for user data management and access.

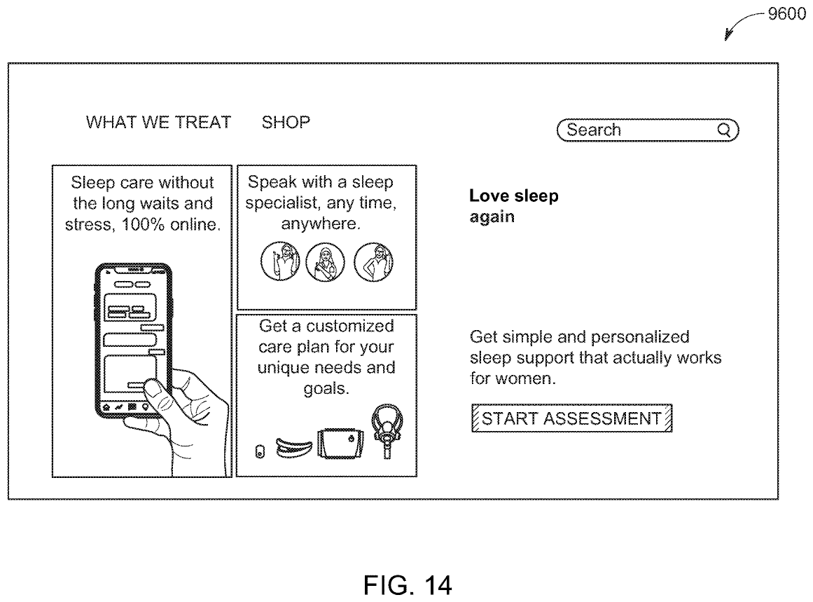

FIG. 14 is a screen image of a web portal providing a virtual sleep pathway for women.

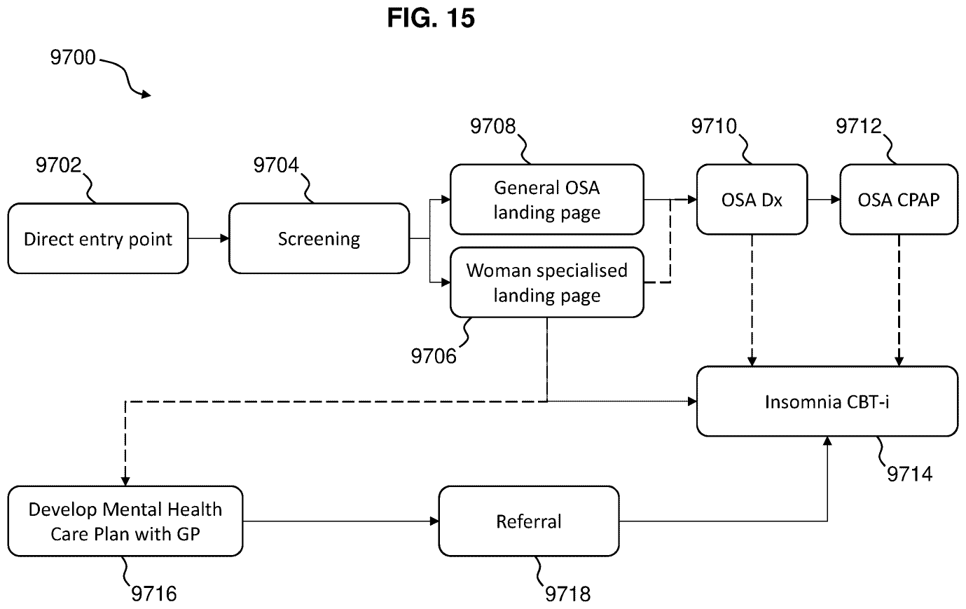

FIG. 15 is a flow chart illustrating a virtual sleep pathway in accordance with one form of the present technology.

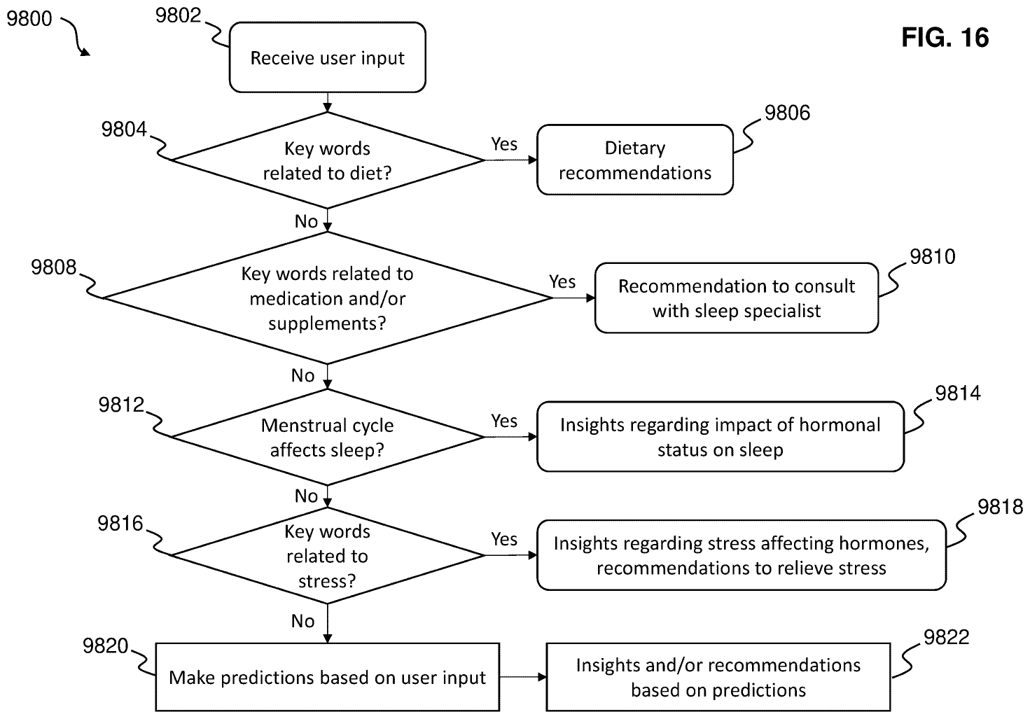

FIG. 16 is a flow chart illustrating a method for provision of sleep related insights and/or recommendations in accordance with one form of the present technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may be suitable for delivering any type of respiratory therapy including, but not limited to, continuous positive airway pressure (CPAP) therapy, non-invasive ventilation (NIV), invasive ventilation (IV), high flow therapy (HFT), oxygen concentration and ventilation.

The respiratory therapy system may comprise an RPT device 4000 for supplying a flow of air to the patient 1000 via an air circuit 4170 and a patient interface 3000 or 3800.

### 5.3 Patient Interface

A non-invasive patient interface 3000 in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

An unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. The air to the nasal prongs may be delivered by one or more air supply lumens 3820a, 3820b that are coupled with the nasal cannula 3800. The lumens 3820a, 3820b lead from the nasal cannula 3800 to a respiratory therapy device via an air circuit. The unsealed patient interface 3800 is particularly suitable for delivery of flow therapies, in which the RPT device generates the flow of air at controlled flow rates rather than controlled pressures. The “vent” at the unsealed patient interface 3800, through which excess airflow escapes to ambient, is the passage between the end of the prongs 3810a and 3810b of the cannula 3800 via the patient's nares to atmosphere.

### 5.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 6 cmH₂O, or at least 10cmH₂O, or at least 20 cmH₂O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors 4272 and flow rate sensors 4274. One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 5.4.1 RPT Device Mechanical & Pneumatic Components

An RPT device may comprise one or more of the following components in an integral unit. In an alternative form, one or more of the following components may be located as respective separate units.

### 5.4.1.1 Air Filter(s)

An RPT device in accordance with one form of the present technology may include an air filter 4110, or a plurality of air filters 4110. In one form, an inlet air filter 4112 is located at the beginning of the pneumatic path upstream of a pressure generator 4140. In one form, an outlet air filter 4114, for example an antibacterial filter, is located between an outlet of the pneumatic block 4020 and a patient interface 3000 or 3800.

### 5.4.1.2 Muffler(s)

An RPT device in accordance with one form of the present technology may include a muffler 4120, or a plurality of mufflers 4120. In one form of the present technology, an inlet muffler 4122 is located in the pneumatic path upstream of a pressure generator 4140. In one form of the present technology, an outlet muffler 4124 is located in the pneumatic path between the pressure generator 4140 and a patient interface 3000 or 3800.

### 5.4.1.3 Pressure or Flow Generator

In certain forms of the technology, the RPT device 4000 comprises a pressure generator or flow generator 4140. In one form of the present technology, a pressure or flow generator 4140 for producing a flow, or a supply, of air at positive pressure is a controllable blower 4142. For example the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH₂O to about 20 cmH₂O, or in other forms up to about 30 cmH₂O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 is under the control of the therapy device controller 4240.

In other forms, a pressure generator 4140 may be a piston-driven pump, a pressure regulator connected to a high pressure source (e.g. compressed air reservoir), or a bellows.

### 5.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000 or 3800. In examples, the one or more transducers 4270 may comprise a flow rate sensor 4274 (e.g. based on a differential pressure transducer, for example, an SDP600 Series differential pressure transducer from SENSIRION), a pressure sensor 4272 located in fluid communication with the pneumatic path (for example, a transducer from the HONEYWELL ASDX series, or a transducer from the NPA Series from GENERAL ELECTRIC), and/or a motor speed transducer 4276 used to determine a rotational velocity of the motor 4144 and/or the blower 4142 (for example, a speed sensor, such as a Hall effect sensor). In other examples the one or more transducers 4270 may comprise an acoustic sensor (e.g. a microphone) and/or an optical sensor (e.g. a camera or barcode reader).

In one form, a signal from a transducer 4270 may be filtered, such as by low-pass, high-pass or band-pass filtering.

### 5.4.2 RPT Device Electrical Components

### 5.4.2.1 Power Supply

A power supply 4210 may be located internal or external of the external housing 4010 of the RPT device 4000. In one form of the present technology, power supply 4210 provides electrical power to the RPT device 4000 only. In another form of the present technology, power supply 4210 provides electrical power to both RPT device 4000 and humidifier 5000.

### 5.4.2.2 Input Devices

In one form of the present technology, an RPT device 4000 includes one or more input devices 4220 in the form of buttons, switches or dials to allow a person (for example a patient or a clinician) to interact with the device. The buttons, switches or dials may be physical devices, or software devices accessible via a touch screen. The buttons, switches or dials may, in one form, be physically connected to the external housing 4010. In one form of the technology an input device 4220 may take the form of a keypad or keyboard with buttons enabling a user to enter a string of characters, for example a series of alphanumeric characters. The keypad may be formed of physical buttons or regions of a touch screen device visually displayed as buttons, or a combination of such buttons.

In other forms, an input device 4220 may take the form of a remote external device 4286 and/or a local external device 4288 separate, or separable, from the RPT device 4000 and in wireless communication with a data communication interface 4280 of the RPT device 4000 that is in electrical connection to the central controller 4230. Exemplary types of wireless communication between the remote external device 4286 and/or a local external device 4288 and the data communication interface 4280 are stated further below.

In one form of the technology, the input device 4220 is a mobile computing device, for example a mobile phone. The mobile computing device may be operable to communicate directly or indirectly with the central controller 4230, for example via an intermediate communication device and/or via data communication interface 4280. The mobile computing device may be configured to run one or more software applications, or apps, that cause one or more graphical user interfaces (GUIs) to be displayed to a user on a screen of the mobile computing device.

In one form, the input device 4220 may be constructed and arranged to allow a person to select a value and/or a menu option.

In certain forms of the technology, one or more transducers 4270 may operate as input devices 4220 enabling information to be sent to central controller 4230. For example, information may be received acoustically (e.g. via multi-frequency signalling) and this information may be input to the RPT device 4000 by detection of the acoustic signal by an acoustic sensor. In another example, information may be received optically (e.g. via barcode, QR code or coded flashing light) and this information may be input to the RPT device 4000 by detection of the optical signal by an optical sensor. It will be appreciated that the data communication interface 4280 may also comprise one or more transducers 4270 (e.g. antennae) and may act as another input device 4220 by which information can be sent to the central controller 4230.

The input devices 4220 are configured to generate signals representative of information or data input by a user and to send the signals to the central controller 4230. For example, the signals may be electrical signals sent along wired connections to the central controller 4230. Additionally, or alternatively, the signals may be wireless communication signals. In one form of the technology, a keypad generates data representative of a character string entered by a user into the keypad and sends data representative of the character string to the central controller 4230.

### 5.4.2.3 Central Controller

In one form of the present technology, the central controller 4230 is one or a plurality of processors suitable to control an RPT device 4000. Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit. In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and the humidifier 5000. The central controller 4230 may be configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300 expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.4.2.4 Therapy Device Controller

In one form of the present technology, therapy device controller 4240 is a virtual controller in the form of therapy control module 4330 that forms part of the algorithms 4300 executed by the central controller 4230. In one form of the present technology, therapy device controller 4240 is a dedicated motor control integrated circuit. For example, in one form a MC33035 brushless DC motor controller, manufactured by ONSEMI is used.

### 5.4.2.5 Memory

In accordance with one form of the present technology the RPT device 4000 includes memory 4260, e.g., non-volatile memory. In some forms, memory 4260 may include battery powered static RAM. In some forms, memory 4260 may include volatile RAM. Memory 4260 may be located on the PCBA 4202. Memory 4260 may be in the form of EEPROM, or NAND flash.

Additionally or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300.

### 5.4.2.6 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g. via Ethernet, or optical fibre) or a wireless protocol (e.g. CDMA, GSM, LTE) to connect to the Internet. In one form, local external communication network 4284 utilises one or more communication standards, such as Bluetooth, Near-Field Communication (NFC), or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician. The local external device 4288 may be a personal computer, mobile computing device (for example a mobile phone or tablet) or remote control.

### 5.4.2.7 Output Devices Including Optional Display, Alarms

In forms of the technology, the RPT device 4000 includes one or more output devices 4290.

An output device 4290 in accordance with the present technology may take the form of one or more of a visual, audio and haptic unit. A visual display may be a Liquid Crystal Display (LCD) or Light Emitting Diode (LED) display.

In forms of the technology the output device 4290 may be comprised as part of a remote external device 4286 and/or a local external device 4288. For example, the output device 4290 may be a display on a mobile computing device (for example a mobile phone or tablet) in wireless communication with the central controller 4230. The mobile computing device may be configured to run one or more software applications, or apps, that cause information to be output on a screen of the mobile computing device.

Data communication interface 4280 may operate as another form of output device 4290 since it may enable information to be output from the RPT device 4000.

### 5.4.3 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 5.4.3.1 Pre-Processing Module

A pre-processing module 4310 in accordance with one form of the present technology receives as an input a signal from a transducer 4270, for example a flow rate sensor 4274 or pressure sensor 4272, and performs one or more process steps to calculate one or more output values that will be used as an input to another module, for example a therapy engine module 4320.

In one form of the present technology, the output values include the interface pressure Pm, the respiratory flow rate Qr, and the leak flow rate Ql.

In various forms of the present technology, the pre-processing module 4310 comprises one or more of the following algorithms: interface pressure estimation 4312, vent flow rate estimation 4314, leak flow rate estimation 4316, and respiratory flow rate estimation 4318.

### 5.4.3.2 Therapy Engine Module

In one form of the present technology, a therapy engine module 4320 receives as inputs one or more of a pressure, Pm, in a patient interface 3000 or 3800, and a respiratory flow rate of air to a patient, Qr, and provides as an output one or more therapy parameters.

In one form of the present technology, a therapy parameter is a treatment pressure Pt.

In one form of the present technology, therapy parameters are one or more of an amplitude of a pressure variation, a base pressure, and a target ventilation.

In various forms, the therapy engine module 4320 comprises one or more of the following algorithms: phase determination 4321, waveform determination 4322, ventilation determination 4323, inspiratory flow limitation determination 4324, apnea/hypopnea determination 4325, snore determination 4326, airway patency determination 4327, target ventilation determination 4328, and therapy parameter determination 4329.

### 5.4.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of air whose interface pressure Pm at the patient interface 3000 or 3800 is equal to the treatment pressure Pt.

### 5.4.3.4 Engine and Control Module for Other Operating Parameters

It has been explained that the central controller 4230 may be configured to implement one or more algorithms 4300 for controlling delivery of respiratory therapy, the algorithms being grouped into a pre-processing module 4310, a therapy engine module 4320 and a therapy control module 4330. The central controller 4230 may additionally, or alternatively, be configured to implement one or more algorithms 4300 for controlling other aspects of the operation of the RPT device 4000. The one or more algorithms 4300 for controlling other aspects of the operation of the RPT device 4000 may be grouped into a pre-processing module, an operation engine module and an operation control module.

### 5.4.3.5 Detection of Fault Conditions

In one form of the present technology, the central controller 4230 executes one or more methods 4340 for the detection of fault conditions, for example, power failure (no power, or insufficient power), transducer fault detection, failure to detect the presence of a component, operating parameters outside recommended ranges (e.g. pressure, flow rate, temperature, PaO₂), and failure of a test alarm to generate a detectable alarm signal.

Upon detection of the fault condition, the corresponding algorithm 4340 signals the presence of the fault by one or more of the following: initiation of an audible, visual &/or kinetic (e.g. vibrating) alarm, sending a message to an external device, and logging of the incident.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800. In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used. In some forms, the air circuit 4170 may comprise one or more heating elements configured to heat air in the air circuit, for example to maintain or raise the temperature of the air. The heating element may be in communication with a controller such as a central controller 4230.

### 5.5.1 Supplementary Gas Delivery

In one form of the present technology, supplementary gas, e.g. oxygen, 4180 is delivered to one or more points in the pneumatic path, such as upstream of the pneumatic block 4020, to the air circuit 4170, and/or to the patient interface 3000 or 3800.

### 5.6 Humidifier

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240. In one form, the humidifier 5000 may comprise a humidifier reservoir dock 5130 (as shown in FIG. 5B) configured to receive the humidifier reservoir 5110.

The water reservoir 5110 may be configured to hold, or retain, a volume of liquid (e.g. water) to be evaporated for humidification of the flow of air. The water reservoir 5110 may be configured to hold a predetermined maximum volume of water in order to provide adequate humidification for at least the duration of a respiratory therapy session, such as one evening of sleep. Typically, the reservoir 5110 is configured to hold several hundred millilitres of water, e.g. 300 millilitres (ml), 325 ml, 350 ml or 400 ml. In other forms, the humidifier 5000 may be configured to receive a supply of water from an external water source such as a building's water supply system.

The humidifier 5000 may comprise one or more humidifier transducers (sensors) 5210 instead of, or in addition to, transducers 4270 described above. Humidifier transducers 5210 may include one or more of an air pressure sensor 5212, an air flow rate transducer 5214, a temperature sensor 5216, or a humidity sensor 5218 as shown in FIG. 5C. A humidifier transducer 5210 may produce one or more output signals which may be communicated to a controller such as the central controller 4230 and/or the humidifier controller 5250. In some forms, a humidifier transducer may be located externally to the humidifier 5000 (such as in the air circuit 4170) while communicating the output signal to the controller.

A heating element 5240 may be provided to the humidifier 5000 in some cases to provide a heat input to one or more of the volume of water in the humidifier reservoir 5110 and/or to the flow of air.

According to one arrangement of the present technology, a humidifier 5000 may comprise a humidifier controller 5250 as shown in FIG. 5C. In one form, the humidifier controller 5250 may be a part of the central controller 4230. In another form, the humidifier controller 5250 may be a separate controller, which may be in communication with the central controller 4230.

In one form, the humidifier controller 5250 may receive as inputs measures of properties (such as temperature, humidity, pressure and/or flow rate), for example of the flow of air, the water in the reservoir 5110 and/or the humidifier 5000. The humidifier controller 5250 may also be configured to execute or implement humidifier algorithms and/or deliver one or more output signals.

As shown in FIG. 5C, the humidifier controller 5250 may comprise one or more controllers, such as a central humidifier controller 5251, a heated air circuit controller 5254 configured to control the temperature of a heated air circuit 4171 and/or a heating element controller 5252 configured to control the temperature of a heating element 5240.

### 5.7 Breathing Waveforms

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak-0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 5.8 Screening, Diagnosis, Monitoring Systems

### 5.8.1 Polysomnography

FIG. 7A shows a patient 1000 undergoing polysomnography (PSG). A PSG system comprises a headbox 2000 which receives and records signals from the following sensors: an EOG electrode 2015; an EEG electrode 2020; an ECG electrode 2025; a submental EMG electrode 2030; a snore sensor 2035; a respiratory inductance plethysmogram (respiratory effort sensor) 2040 on a chest band; a respiratory inductance plethysmogram (respiratory effort sensor) 2045 on an abdominal band; an oro-nasal cannula 2050 with oral thermistor; a photoplethysmograph (pulse oximeter) 2055; and a body position sensor 2060. The electrical signals are referred to a ground electrode (ISOG) 2010 positioned in the centre of the forehead.

### 5.8.2 Non-Obtrusive Monitoring System

One example of a monitoring apparatus 7100 for monitoring the respiration of a sleeping patient 1000 is illustrated in FIG. 7B. The monitoring apparatus 7100 contains a contactless motion sensor generally directed toward the patient 1000. The motion sensor is configured to generate one or more signals representing bodily movement of the patient 1000, from which may be obtained a signal representing respiratory movement of the patient.

### 5.8.3 Respiratory Polygraphy

Respiratory polygraphy (RPG) is a term for a simplified form of PSG without the electrical signals (EOG, EEG, EMG), snore, or body position sensors. RPG comprises at least a thoracic movement signal from a respiratory inductance plethysmogram (movement sensor) on a chest band, e.g. the movement sensor 2040, a nasal pressure signal sensed via a nasal cannula, and an oxygen saturation signal from a pulse oximeter, e.g. the pulse oximeter 2055. The three RPG signals, or channels, are received by an RPG headbox, similar to the PSG headbox 2000.

In certain configurations, a nasal pressure signal is a satisfactory proxy for a nasal flow rate signal generated by a flow rate transducer in-line with a sealed nasal mask, in that the nasal pressure signal is comparable in shape to the nasal flow rate signal. The nasal flow rate in turn is equal to the respiratory flow rate if the patient's mouth is kept closed, i.e. in the absence of mouth leaks.

FIG. 7C is a block diagram illustrating a screening/diagnosis/monitoring device 7200 that may be used to implement an RPG headbox in an RPG screening/diagnosis/monitoring system. The screening/diagnosis/monitoring device 7200 receives the three RPG channels mentioned above (a signal indicative of thoracic movement, a signal indicative of nasal flow rate, and a signal indicative of oxygen saturation) at a data input interface 7260. The screening/diagnosis/monitoring device 7200 also contains a processor 7210 configured to carry out encoded instructions. The screening/diagnosis/monitoring device 7200 also contains a non-transitory computer readable memory/storage medium 7230.

Memory 7230 may be the screening/diagnosis/monitoring device 7200's internal memory, such as RAM, flash memory or ROM. In some implementations, memory 7230 may also be a removable or external memory linked to screening/diagnosis/monitoring device 7200, such as an SD card, server, USB flash drive or optical disc, for example. In other implementations, memory 7230 can be a combination of external and internal memory. Memory 7230 includes stored data 7240 and processor control instructions (code) 7250 adapted to configure the processor 7210 to perform certain tasks. Stored data 7240 can include RPG channel data received by data input interface 7260, and other data that is provided as a component part of an application. Processor control instructions 7250 can also be provided as a component part of an application program. The processor 7210 is configured to read the code 7250 from the memory 7230 and execute the encoded instructions. In particular, the code 7250 may contain instructions adapted to configure the processor 7210 to carry out methods of processing the RPG channel data provided by the interface 7260. One such method may be to store the RPG channel data as data 7240 in the memory 7230. Another such method may be to analyse the stored RPG data to extract features. The processor 7210 may store the results of such analysis as data 7240 in the memory 7230.

The screening/diagnosis/monitoring device 7200 may also contain a communication interface 7220. The code 7250 may contain instructions configured to allow the processor 7210 to communicate with an external computing device via the communication interface 7220. The mode of communication may be wired or wireless. In one such implementation, the processor 7210 may transmit the stored RPG channel data from the data 7240 to the remote computing device. In such an implementation, the remote computing device may be configured to analyse the received RPG data to extract features. In another such implementation, the processor 7210 may transmit the analysis results from the data 7240 to the remote computing device.

Alternatively, if the memory 7230 is removable from the screening/diagnosis/monitoring device 7200, the remote computing device may be configured to be connected to the removable memory 7230. In such an implementation, the remote computing device may be configured to analyse the RPG data retrieved from the removable memory 7230 to extract the features.

### 5.9 Portable Oxygen Concentrator

Oxygen concentrators may take advantage of pressure swing adsorption (PSA). Pressure swing adsorption may involve using a compressor to increase gas pressure inside a canister that contains particles of a gas separation adsorbent. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The gas separation adsorbent may be regenerated by reducing the pressure, which reverses the adsorption of molecules from the adsorbent. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

FIG. 8A illustrates a schematic diagram of an oxygen concentrator 8000, according to an implementation. Oxygen concentrator 8000 may concentrate oxygen out of an air stream to provide oxygen enriched gas to a user. Oxygen concentrator 8000 may be a portable oxygen concentrator. For example, oxygen concentrator 8000 may have a weight and size that allows the oxygen concentrator to be carried by hand and/or in a carrying case.

Oxygen may be collected from ambient air by pressurising ambient air in canisters 8100, including first canister 8102 and 8104, which include a gas separation adsorbent. Gas separation adsorbents useful in an oxygen concentrator are capable of separating at least nitrogen from an air stream to produce oxygen enriched gas. As shown in FIG. 8B, air may be drawn into the oxygen concentrator 8000 through air inlet 8002 by compression system 8200. Compression system 8200 may draw in air from the surroundings of the oxygen concentrator and compress the air, forcing the compressed air into one or both of canisters 8102 and 8104. Compression system 8200 may include one or more compressors capable of compressing air. In an implementation, an inlet muffler 8004 may be coupled to air inlet 8002 to reduce sound produced by air being pulled into the oxygen concentrator by compression system 8200.

Coupled to each canister 8102/8104 are inlet valves 8020/8022 and outlet valves 8030/8032. As shown in FIG. 8B, inlet valves 8020/8022 are used to control the passage of air from compression system 8200 to the respective canisters. Outlet valves 8030/8032 are used to release gas from the respective canisters during a venting process. In an implementation, pressurized air is sent into one of canisters 8102 or 8104 while the other canister is being vented.

In an implementation, a controller 8300 is electrically coupled to valves 8020, 8022, 8030, and 8032. Controller 8300 includes one or more processors 8310 operable to execute program instructions stored in memory 8320. The program instructions are operable to perform various predefined methods that are used to operate the oxygen concentrator 8000, such as the methods described in more detail herein. Controller 8300 may include program instructions for operating inlet valves 8020 and 8022 out of phase with each other, i.e., when one of inlet valves 8020 or 8022 is opened, the other valve is closed. During pressurization of canister 8102, outlet valve 8030 is closed and outlet valve 8032 is opened. Similar to the inlet valves, outlet valves 8030 and 8032 are operated out of phase with each other. In some implementations, the voltages and the duration of the voltages used to open the input and output valves may be controlled by controller 8300.

Check valves 8040 and 8042 are coupled to canisters 8102 and 8104, respectively. Check valves 8040 and 8042 are one-way valves that are passively operated by the pressure differentials that occur as the canisters are pressurized and vented. Check valves 8040 and 8042 are coupled to canisters to allow oxygen produced during pressurization of the canister to flow out of the canister, and to inhibit back flow of oxygen or any other gases into the canister.

Under pressure, nitrogen molecules in the pressurized ambient air are adsorbed by the gas separation adsorbent in the pressurized canister. As the pressure increases, more nitrogen is adsorbed until the gas in the canister is enriched in oxygen. The non-adsorbed gas molecules (mainly oxygen) flow out of the pressurized canister when the pressure reaches a point sufficient to overcome the resistance of the check valve coupled to the canister. In an exemplary implementation, canister 8102 is pressurized by compressed air produced in compression system 8200 and passed into canister 8102, and canister 8104 is vented substantially simultaneously while canister 8102 is pressurized. Canister 8102 is pressurized until the pressure in canister is sufficient to open check valve 8040. Oxygen enriched gas produced in canister 8102 exits through check valve and, in one implementation, is collected in accumulator 8006.

After some time, the gas separation adsorbent will become saturated with nitrogen and will be unable to separate significant amounts of nitrogen from incoming air. This point is usually reached after a predetermined time of oxygen enriched gas production. In the implementation described above, when the gas separation adsorbent in canister 8102 reaches this saturation point, the inflow of compressed air is stopped and canister 8102 is vented to remove nitrogen. While canister 8102 is being vented, canister 8104 is pressurized to produce oxygen enriched gas in the same manner described above. Pressurization of canister 8104 is achieved by closing outlet valve 8032 and opening inlet valve 8022. The oxygen enriched gas exits canister 8104 through check valve 8042.

During venting of canister 8102, outlet valve 8030 is opened allowing pressurized gas (mainly nitrogen) to exit the canister through concentrator outlet 8008. In an implementation, the vented gases may be directed through muffler 8010 to reduce the noise produced by releasing the pressurized gas from the canister. As gas is released from canister 8102, the pressure in the canister drops, allowing the nitrogen to become desorbed from the gas separation adsorbent. The released nitrogen exits the canister through outlet 8008, resetting the canister to a state that allows renewed separation of oxygen from an air stream.

During venting of the canisters, it is advantageous that at least a majority of the nitrogen is removed. In some implementations, a canister may be further purged of nitrogen using an oxygen enriched stream that is introduced into the canister from the other canister. In an exemplary implementation, a portion of the oxygen enriched gas may be transferred from canister 8102 to canister 8104 when canister 8104 is being vented of nitrogen. In an implementation, oxygen enriched gas may travel through flow restrictors 8050, 8052, and 8054 between the two canisters.

Flow of oxygen enriched gas is also controlled by use of valve 8056 and valve 8058. Valves 8056 and 8058 may be opened for a duration during the venting process (and may be closed otherwise) to prevent excessive oxygen loss out of the purging canister. In an exemplary implementation, when canister 8102 is being vented it is desirable to purge canister 8102 by passing a portion of the oxygen enriched gas being produced in canister 8104 into canister 8102. A portion of oxygen enriched gas, upon pressurization of canister 8104, will pass through flow restrictor 8050 into canister 8102 during venting of canister 8102. Additional oxygen enriched air is passed into canister 8102, from canister 8104, through valve 8058 and flow restrictor 8054. Valve 8056 may remain closed during the transfer process, or may be opened if additional oxygen enriched gas is needed. The selection of appropriate flow restrictors 8050 and 8054, coupled with controlled opening of valve 8058 allows a controlled amount of oxygen enriched gas to be sent from canister 8104 to 8102. In an implementation, the controlled amount of oxygen enriched gas is an amount sufficient to purge canister 8102 and minimize the loss of oxygen enriched gas through venting valve 8030 of canister 8102. While this implementation describes venting of canister 8102, it should be understood that the same process can be used to vent canister 8104 using flow restrictor 8050, valve 8056 and flow restrictor 8052.

The pair of equalization/vent valves 8056/8058 work with flow restrictors 8052 and 8054 to optimize the air flow balance between the two canisters. In some implementations, the air pathway may not have restrictors but may instead have a valve with a built-in resistance or the air pathway itself may have a narrow radius to provide resistance.

At times, oxygen concentrator may be shut down for a period of time. In an implementation, outside air may be inhibited from entering canisters after the oxygen concentrator is shutdown by pressurising both canisters prior to shutdown. In an implementation, the pressure in the canisters, at shutdown, should be at least greater than ambient pressure. As used herein the term “ambient pressure” refers to the pressure of the surroundings in which the oxygen concentrator is located (e.g. the pressure inside a room, outside, in a plane, etc.). In an implementation, the pressure in the canisters, at shutdown, is at least greater than standard atmospheric pressure (i.e., greater than 760 mmHg (Torr), 1 atm, 101,325 Pa).

FIG. 8B depicts an example of the portable oxygen concentrator 8000. In this implementation the oxygen concentrator 8000 includes an outer housing 8500. Outer housing 8500 includes compression system inlets 8502, cooling system passive inlet 8504 (not seen in FIG. 8B, but indicated by reference numeral 8502) and cooling system passive outlet 8504 at each end of outer housing 8500. The outer housing 8500 also includes outlet port 8506, and a control panel 8600. Inlet 8502 and outlet 8504 allow cooling air to enter the housing 8500, flow through the housing, and exit the interior of housing to aid in cooling of the oxygen concentrator 8000. Compression system inlets 8502 allow air to enter the compression system 8200 shown in FIG. 8A). Outlet port 8506 is used to attach a conduit to provide oxygen enriched gas produced by the oxygen concentrator 8000 to a user. Control panel 8600 serves as an interface between a user and controller 8300 (shown in FIG. 8A) to allow the user to initiate predetermined operation modes of the oxygen concentrator 8000 and to monitor the status of the system. Charging input port 8602 may be disposed in control panel 8600.

### 5.10 Respiratory Therapy Modes

Various respiratory therapy modes may be implemented by the disclosed respiratory therapy system. Examples of the respiratory therapy modes may include CPAP therapy, bi-level therapy, and high flow therapy.

### 5.11 Computing System and Processes

In forms of the technology, the RPT device 4000 may be part of, or may operate in conjunction with, a system 9000. System 9000 may comprise one or more servers 9010 and one or more computing devices 9040, and may generally be referred to as a computing system 9000. Components of system 9000 may interact with RPT device 4000, for example to control and/or monitor operation of the RPT device 4000. In some examples, system 9000 may enable a person (e.g. a patient, a clinician) to control and/or monitor operation of the RPT device 4000. Controlling and/or monitoring operation of the RPT device 4000 may enable the respiratory therapy provided to the patient 1000 to be controlled and/or monitored.

### 5.11.1 Computing System

FIG. 9 depicts an example system 9000 that may be implemented for monitoring sleep and providing insights and/or recommendations. The system 9000 may generally include one or more of servers 9010, one or more communication networks 9030, and one or more computing devices 9040. The server 9010 and computing device 9040 may also be in communication with one or more respiratory therapy devices (for example, but not limited to, the RPT device 4000 described in relation to FIG. 4A to FIG. 4E above) via the one or more communication networks 9030.

The one or more communication networks 9030 may comprise, for example, the Internet, a local area network, a wide area network and/or a personal area network implemented over wired communication network(s) 9032, wireless communication network(s) 9034, or a combination thereof (for example, a wired network with a wireless link). In one form, local communication networks may utilize one or more communication standards, such as Bluetooth, Near-Field Communication (NFC), or a consumer infrared protocol.

The server 9010 may comprise processing facilities represented by one or more processors 9012, memory 9014, and other components typically present in such computing environments. The processing capabilities of the processor 9012 may be provided, for example, by one or more general-purpose processors, one or more special-purpose processors, or cloud computing services providing access to a shared pool of computing resources configured in accordance with desired characteristics, service models, and deployment models. In the example illustrated the memory 9014 stores information accessible by processor 9012, the information including instructions 9016 that may be executed by the processor 9012 and data 9018 that may be retrieved, manipulated or stored by the processor 9012. The memory 9014 may be of any suitable means known in the art, capable of storing information in a manner accessible by the processor 9012, including a computer readable medium, or other medium that stores data that may be read with the aid of an electronic device. Although the processor 9012 and memory 9014 are illustrated as being within a single unit, it should be appreciated that this is not intended to be limiting, and that the functionality of each as herein described may be performed by multiple processors and memories, that may or may not be remote from each other and the remainder of system 9000.

The instructions 9016 may include any set of instructions suitable for execution by the processor 9012. For example, the instructions 9016 may be stored as computer code on the computer readable medium. The instructions may be stored in any suitable computer language or format. Data 9018 may be retrieved, stored or modified by processor 9012 in accordance with the instructions 9016. The data 9018 may also be formatted in any suitable computer readable format. Again, while the data is illustrated as being contained at a single location, it should be appreciated that this is not intended to be limiting—the data may be stored in multiple memories or locations. The data 9018 may include one or more databases 9020.

In some examples, the server 9010 may communicate one-way with computing device(s) 9040 by providing information to one or more of the computing devices 9040, or vice versa. In other embodiments, server 9010 and computing device(s) 9040 may communicate with each other two-way and may share information and/or processing tasks.

In some examples, the computing device(s) 9040 may include the remote external device 4286 and/or the local external device 4288 described with reference to FIG. 4C above.

### 5.11.2 Computing Devices

The computing device(s) 9040 can be any suitable processing device such as, without limitation, a personal computer such as a desktop or laptop computer 9042, or a mobile computing device such as a smartphone 9044 or tablet 9046. FIG. 10 depicts an exemplary general architecture 9100 of a computing device 9040. Computing device 9040 may include one or more processors 9110. Computing device 9040 may also include memory/data storage 9120, input/output (I/O) devices 9130, and communication interface 9150.

The one or more processors 9110 can include functional components used in the execution of instructions, such as functional components to fetch control instructions from locations such as memory/data storage 9120, decode program instructions, and execute program instructions, and write results of the executed instructions.

Memory/data storage 9120 may be the computing device's internal memory, such as RAM, flash memory or ROM. In some examples, memory/data storage 9120 may also be external memory linked to computing device 9040, such as an SD card, USB flash drive, optical disc, or a remotely located memory (e.g. accessed via a server such as server 9010), for example. In other examples, memory/data storage 9120 can be a combination of external and internal memory.

Memory/data storage 9120 includes processor control instructions 9122 and stored data 9124 that instruct processor 9110 to perform certain tasks, as described herein. As noted above, in examples instructions may be executed by, and data stored in and/or accessed from, resources associated with the server 9010 in communication with the computing device 9040.

In examples, the input/output (I/O) devices 9130 may include one or more displays 9132. In examples, the display 9132 may be a touch sensitive screen allowing for user input in addition to outputting visible information to a user of computing device 9030. In examples, I/O devices may include other output devices, including one or more speakers 9134, and haptic feedback devices 9136. In examples, the input/output (I/O) devices 9130 may include input devices such as physical input devices 9138 (for example, buttons or switches), optical sensors 9140 (for example, one or more imaging devices such as a camera), and inertial sensors 9142 (particularly in examples where the computing device 9040 is a mobile computing device). It will be appreciated that other I/O devices 9130 may be included, or otherwise accessed through an I/O interface 9150 (for example, interfacing with peripheral devices connected to the computing device 9040). A communication interface 9160 enables computing device 9040 to communicate via the one or more networks 9030 (shown in FIG. 9).

### 5.11.3 Computer-Implementable Methods

Computer readable instructions may implement the exemplary methods described herein. In examples, the computer readable instructions comprise one or more algorithms for execution by one or more of the processors, for example processors 9012 and/or central controller 4230, described herein. The instructions for performing these functions are, optionally, included in a non-transitory computer readable storage medium, for example memory 9014, or other computer program product configured for execution by one or more processors. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A computer readable storage medium, as used herein, is not to be construed as being transitory signals per se, such as radio waves or other freely propagating electromagnetic waves, electromagnetic waves propagating through a waveguide or other transmission media, or electrical signals transmitted through a wire.

However, persons of ordinary skill in the art will readily appreciate that the entire algorithm and/or parts thereof can alternatively be executed by a device other than a processor and/or embodied in firmware or dedicated hardware in a well-known manner, e.g., it may be implemented by an application specific integrated circuit (ASIC), a programmable logic device (PLD), a field programmable logic device (FPLD), a field programmable gate array (FPGA), discrete logic, etc. For example, any or all of the components can be implemented by software, hardware, and/or firmware. Also, some or all of the instructions represented by the flowcharts may be implemented manually. Further, although the example algorithms are described with reference to the illustrated flowcharts, persons of ordinary skill in the art will readily appreciate that many other methods of implementing the example processor readable instructions may alternatively be used. For example, the order of execution of the blocks may be changed, and/or some of the blocks described may be changed, eliminated, or combined.

As used herein the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor, a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a processor readable medium; or a combination thereof.

### 5.12 Systems and Processes for Promoting Sleep

Examples of the present technology disclose a system to assist with promoting sleep for individuals, with consideration given to the influence of factors that are particular to women-especially relating to hormonal status. Hormonal fluctuations are prevalent during the menstrual cycle, but such fluctuations also occur due to a range of influences and conditions. The system may receive inputs from one or more sensors, and/or manually input by the user into an interface, and determine personalized recommendations and/or insights to help improve the user's sleep.

As used herein, the term “insomnia” generally refers to a condition by which a person has persistent problems with falling or staying asleep. It should be appreciated that the term is used herein for ease of understanding, and is not intended to be exclusively equated with a clinical diagnosis or classification of the condition.

### 5.12.1 Data Collection and Processing

### 5.12.1.1 Sensors and Wearable Devices

In some examples, the system 9000 may include one or more sensors configured to monitor one or more characteristics of a user from which one or more sleep events may be determined. For example, as shown in FIG. 11 the one or more sensors may be an optical sensor of a wearable sensor device 9200 configured to measure a blood volume pulse as described in U.S. Published Patent Application No. 2020/0015737, published Jan. 16, 2020, and entitled “Apparatus, System and Method for Diagnosing Sleep”, which is incorporated herein by reference. Wearable sensor device 9200 is configured to be attached by one side to a tissue of the patient, for example onto a fingertip. At the side, wearable sensor device 9200 comprises a reflectance based optical sensor. This sensor is configured to measure the blood volume pulse when attached to the patient. When attached, the sensor emits light onto the tissue of the patient and measures the reflected light. The measured signal is then indicative for the blood volume pulse in the tissue where the sensor is attached to.

The wearable sensor device 9200 may be configured to measure and wirelessly transmit the blood volume pulse of the patient during sleep—for example to a mobile communication device such as smartphone 9044. In examples, processing of the blood volume pulse data may be performed by smartphone 9044, a remote computing system such as servers 9010, or a combination thereof.

Additional physiological data may be obtained, for example, using a wearable device 9202 that may have computing functions including communication with the smartphone 9044 and/or network 9030. In this example, the wearable device 9202 is a wrist mounted device (examples of which may include an Apple® watch or a Fitbit®). The wearable device 9202 includes one or more physiological sensors that collects physiological data. In this example, the sensor may include heart rate sensors, oxygen level sensors, ECG sensors, pulse rate sensors, and the like. The wearable device 9202 may include other sensors such as one or more of: an accelerometer, a gyroscope, a GPS receiver, a sound sensor and/or microphone.

The one or more sensors may be provided in a range of forms. For example, the one or more sensors may be provided in a headband 9204, ring 9206, or mat 9208 placed under the user while sleeping. In further examples the one or more sensors may be provided in head gear (e.g. eye mask, earing, nose wearable, nose mask, face mask), chest gear (e.g. chest patch, chest strap, necklace, bra or bra insert), and/or other paraphernalia (e.g. wristband, toe ring, glove).

In examples, one or more characteristics of a user may be obtained using a non-contact sensing device as described in, for example, International Patent Publication Nos. WO 2018/050913 and WO 2020/104465, each of which is hereby incorporated by reference herein in its entirety. Further examples of non-contact sensing devices may include the “S+” sleep tracking system by ResMed Pty Ltd, and the “Aura” sleep system by Withings.

### 5.12.1.2 User Input

In some forms of the present technology additional data may be obtained by the system 9000, for example, by a user manually inputting information. For example, a user may input data relating to one or more of: medications, supplements, current symptoms, comorbid conditions (e.g. OSA, depression, bipolarism, diabetes, hypertension, etc.), food, drink, sleep hygiene habits, other physiological experiences (e.g. self-reported temperature, menstrual cycle, illness, night urination frequency), environmental factors (e.g. noise, light, room temperature, bed partner disturbances), change of sleep location (e.g. new house, travel), flight/change of time zone, new or unusual activity, use of medical devices (e.g. CPAP devices), social interactions (e.g. care-giving for another, addition of a family member/visitor to a household, bereavement, loneliness), and user perceived characteristics (e.g. alertness, tiredness, mood, stress and anxiety).

In examples, commonly input forms of data may be selectable (e.g. via tags), with free form text allowing for user entry of unusual or less common factors the user believes could potentially be affecting their sleep (e.g. sports activity, dental visits, stressful social interactions).

### 5.12.1.3 Menstrual Cycle Events

The menstrual cycle includes four phases: menstruation, the follicular phase, ovulation and the luteal phase. Sleep is impacted in different ways across the four phases, for example the risk of sleep disordered breathing varies across the cycle. Sleep disturbances are most pronounced during periods of significant menstrual cycle changes and hormonal fluctuations. For example, polycystic ovary syndrome (PCOS) may result in prolonged cycles, or no cycles, and increase the risk of sleep disordered breathing (SDB) and/or problems with falling or staying asleep (more particularly, insomnia). As a further example: pregnancy, during which there are no cycles, may increase the risk of SDB and/or restless legs syndrome. As a further example: perimenopause or menopause, with highly variable cycles, may increase the risk of SDB and/or insomnia.

Information regarding a woman's menstrual cycle assists in informing assessment of sleep and recommendations for managing and improving same. Rising and falling levels of the hormones estrogen and progesterone, which regulate the menstrual cycle, can affect a woman's ability to fall and stay asleep. Menstrual cycle patterns may reveal a woman's hormonal status and thus aid in the provision of contextual sleep recommendations relevant to their hormonal status at that time.

In addition, no two women are the same. Correlation of an individual's sleep data with their menstrual cycle data may uncover insights on how their sleep changes across their menstrual cycle phases, and allow deeper personalization of sleep recommendations across the cycle phases.

In examples, the user inputs information into the system relating to their menstrual cycle. For example, a user interface can provide the capability for a woman to enter the actual events as they are experienced (e.g. menses, or other symptoms). This allows for regular tracking (e.g. daily) of the information. Examples of such information include: level of tiredness/fatigue (e.g. less, normal, more); instances of localised pain (e.g. headache, stomach pain, breast pain, back pain); period characteristics such as level of bleeding flow (e.g. mild, normal, heavy), pain level (e.g. low, medium, high), associated symptoms (e.g. fatigue, localised pain, cramping, mood); period timing characteristics of current or previous cycle (e.g. start, end, duration, regularity, missed periods); medication impacting menstrual cycle (e.g. to delay onset of period); and/or other factors such as pregnancy, lactation, contraceptives use, perimenopause, instances of hot flashes, nausea, constipation, acne, mood, and/or appetite.

In examples, menstrual cycle information may be determined using one or more characteristics of a user obtained using one or more sensors. Methods for predicting timing of events related to a woman's menstrual cycle are described in, for example, US Patent Publication No. 2020/0000441 and International Patent Publication No. WO 2021/263209, each of which is hereby incorporated by reference herein in its entirety.

### 5.12.1.4 Determining Sleep Events, States, and Stages

Peripheral arterial tone may be derived from the measured blood volume pulse obtained using wearable sensor device 9200, e.g. by deriving the envelope of the obtained blood volume pulse measurements, more particularly the upper envelope and lower envelope of the measured blood volume pulse. As the measured blood volume pulse is an oscillating signal with a periodicity determined by the heartbeat of the patient, the upper envelope is determined as a smooth curve outlining the upper extremities of the measured blood volume pulse and the lower envelope is determined by the lower extremities of the measured blood volume pulse. The difference between the upper and lower envelopes is then indicative of the peripheral arterial tone.

One or more sleep events are determined by inspecting the changes in the peripheral arterial tone, e.g. by inspecting the decrease in the amplitude of the peripheral arterial tone and/or the increase in the amplitude of the peripheral arterial tone, optionally in combination with the measured blood volume pulse. A sleep event may for example comprise a sleep disordered breathing events such as apnoeic events, periods of intense snoring, limb movements either as periodic or single events, cortical arousals, autonomic arousals, periods of bruxism, hypnic jerks, tossing events, and turning events. For example, an apnoeic event may be determined by considering the temporal proximity of a peripheral arterial tone amplitude decrease, a blood oxygen desaturation, and a decrease of the inter pulse interval of the blood volume pulse. An autonomic arousal may be determined by considering the temporal proximity of a peripheral arterial tone amplitude decrease and a decrease of the inter-pulse-interval of the blood volume pulse. A bruxism event may be determined by the temporal proximity of a peripheral arterial tone amplitude decrease and a decrease of the inter-pulse-interval of the blood volume pulse, under the absence of limb movement. A periodic limb movement event may be determined by the temporal proximity of a peripheral arterial tone amplitude decrease and a decrease of the inter-pulse-interval of the blood volume pulse, under the presence of limb movement.

Methods for determining sleep states and/or sleep stages from physiological data generated by one or more sensors and/or user inputs, and determination of a sleep score, are described in, for example, International Patent Publication No. WO 2014/047310, U.S. Patent Publication No. 2015/0230750, U.S. Patent Publication No. 2014/0088373, U.S. Patent Publication No. 2016/0151603, WO 2017/132726, WO 2018/050913, WO 2019/122413, WO 2019/122414, U.S. Patent Publication No. 2020/383580, WO 2020/104465, and WO/2021/214640, each of which is hereby incorporated by reference herein in its entirety. and

### 5.12.2 User Interface

FIG. 12A illustrates a home interface 9300 of an exemplary user interface displayed, for example, on smartphone 9044. In this example, the home interface 9300 includes a sleep score indicator 9302 displaying the user's latest sleep score in numerical forms and as a pie gauge 9304. In this example a summary recommendation 9306 in text form is presented above the sleep score indicator 9302, providing the user with a summary of current recommendations and/or insights.

In this example the home interface 9300 includes a temperature button 9308, menstrual cycle button 9310, mood button 9312, and focus button 9314. Each of the buttons may include an indicator of a current status or characteristic of the user. For example, temperature button 9308 may include an indication of a trend in temperature increase/decrease, menstrual cycle button 9310 may include an indication of the user's current phase of their menstrual cycle, mood button 9312 may include a mood icon indicating the user's current mood, and focus button 9314 may include an indicator of the user's focus as a percentage (e.g. in numerical form and/or a graphical representation such as a gauge).

In this example the home interface 9300 includes a diary section 9316, displaying user input data regarding one or more categories, for example: medication/supplements taken, current symptoms of the user (e.g. headache, stomach ache, back pain), food and/or drink. Selection of the diary section 9316 may open a diary interface, enabling the user to input information.

In this example the home interface 9300 includes a navigation bar 9318 including home icon 9320, insight icon 9322, chat icon 9324, recommendations icon 9326, and contacts icon 9328. Selection of the respective icons navigates the user to respective interfaces, described further below.

In this example, selection of the sleep score indicator 9302 navigates the user to a sleep diagnosis screen 9330, as shown in FIG. 12B. The sleep diagnosis screen 9330 displays a sleep state bar gauge 9332 visualising the user's time asleep relative to her time in bed. The sleep diagnosis screen 9330 further displays a sleep stage graph 9334 displaying the user's sleep stages over time, for example: awake, REM sleep, deep sleep, and light sleep. A sleep stage section 9336 displays the sleep stages as a bar graph, with numerical values displayed for the time (and percentage relative to total time) in each stage. In this example the sleep diagnosis screen 9330 further includes a breathing pauses graph 9338 displaying the occurrences of breathing pauses over the same time scale as the sleep stage graph 9334. In this example the sleep diagnosis screen 9330 further includes a heart rate line graph 9340 showing the user's heart rate over the course of sleep.

In this example, selection of the menstrual cycle button 9310 navigates the user to a menstrual cycle screen 9350, as shown in FIG. 12C. The menstrual cycle screen 9350 includes a cycle stage indicator 9352, displaying the current stage of the user's menstrual cycle as recorded with system 9000. The menstrual cycle screen 9350 further includes a projected cycle timing section 9354, displaying the projected time until the user reaches the next stage of her menstrual cycle. The menstrual cycle screen 9350 further includes a cycle statistics section 9356 displaying statistics for the user's menstrual cycle during her use of the system 9000. For example, the cycle statistics section 9356 may display one or more of: average cycle length, shortest cycle length, longest cycle width, and cycle length variability.

In this example the menstrual cycle screen 9350 includes a menstrual cycle factors tab 9358. This allows for capture of any physiological factors that the user is aware of that may affect their menstrual cycle. Selecting the “Factors” button will bring up a list of options to select from (e.g. pregnancy, lactation, contraceptives use, PCOS, perimenopause).

In examples, selection of the contacts icon 9328 may lead to a community page where the user may interact with other users. For example, the community page may provide a forum where people may ask questions related to sleep and women's health and other users may respond.

In examples, the system 9000 may initiate interaction with the user to take action to promote sleep—for example establish a program, initiate an established program, or engage with a health professional. For example, on determining that conditions are such that the user is likely to encounter difficulties in falling asleep or achieving a desirable quality of sleep, the system 9000 may issue a notification to the user via the smartphone 9044 (for example an alert displayed on the lock screen, alert centre, or in the application providing the user interface) inviting her to start a program or take another action to assist with remedying this. Referring to FIG. 12D and FIG. 12E, further engagement may be performed via a chat interface 9370. An initiation message 9372 is presented to the user: alerting her to the identified issue (e.g. an approaching change in menstrual cycle stage to one in which she typically has difficulty falling asleep), identifying a potential action (e.g. developing a program to help condition her body for sleep), and providing user selectable actions (e.g. yes, or no). On receiving an indication that the user wishes to engage with the solution, the system may present further queries to the user to assist with customising the solution to her needs. In the example illustrated, a first query message 9374 may request user input of factors inhibiting her from achieving sleep, for example selection of factors such as racing thoughts, anxiety, restless legs, and inability to remain asleep. On receiving the user input, a second query message 9376 may request user input regarding preferred time for falling asleep. The user input 9378 in response may be via any suitable means, e.g. a drop down menu, or text field. On receiving the user input, the system 9000 may establish a personalised sleep program for the user, and present the user the option to view the program in program notification message 9380. Referring to FIG. 12F and FIG. 12G, a program summary interface 9382 is presented to the user-which in examples may be accessed via selection of program notification message 9380 or toggling from the chat interface 9370 using function selector 9384. The program summary interface 9382 displays a current program recommendation 9386, and a previous recommendation section 9388 displaying previously established personalised programs (for example a menopause focus mindfulness program 9388a and a Cognitive behavioural therapy (CBT)/Cognitive behavioural therapy for Insomnia (CBT-i) program 9388b). Selection of the current program recommendation 9386 displays program activity stages (in this example, sleep hygiene practice stage 9390 in which the user is provided with guidance on preparing the user and their sleep environment for sleep, journaling stage 9392 in which the user is encouraged to write down of things they need to remember, and breathing meditation stage 9394 in which the user is guided through breathing exercises), and a program start button 9396.

In examples, the system 9000 may initiate interaction with the user on detection of potential sleep disorders. For example, referring to FIG. 12H and FIG. 12I an issue alert message 9400 is presented to the user: alerting her to an identified issue (e.g. an unusually high number of breathing pauses for multiple consecutive nights) and asking if the user wishes to view additional information. On receiving an indication that the user wishes to view additional information, the system may present a first informational message 9402—in this example an explanation of sleep apnea and how the user's breathing pauses may be indicative of the user suffering from this—and invite the user to consult with a sleep consultant to discuss further. On acceptance of the invitation, the user is navigated to sleep consultant interface 9410 as shown in FIG. 12J and provided with selectable call button 9412 and appointment booking button 9414 to initiate contact with a sleep consultant or sleep health professional.

In examples, the sleep consultant interface 9410 may form part of a recommendations portion of the application. In examples selectable options may be provided to navigate between different interfaces of the recommendations portion, e.g. a relaxation button 9416, learn button 9418, shop button 9420, and consult button 9422. Selection of the learn button 9418 displays learning interface 9424 as shown in FIG. 12K, providing selectable links 9426 to information regarding sleep related issues. Selection of the relaxation button 9416 displays relaxation interface 9428 as shown in FIG. 12L, providing breathing exercise section 9430, stretching exercise section 9432, and meditation exercise section 9434—each including a plurality of selectable exercises in the respective categories for use by the user. Selection of the shop button 9420 displays shop interface 9420 as shown in FIG. 12M, enabling the user to browse and purchase or order various products and/or services—for example sleep aids for insomnia 9438 and CPAP machines and masks 9440.

In examples, the system 9000 provides the user insights into sleep related matters. For example, on selection of the sleep score indicator 9302 in the home interface 9300 a sleep insights interface 9450 is displayed, as shown in FIG. 12N and FIG. 12O. The sleep score insights interface 9450 includes a time scale selector 9452 to enable the user to select the time scale over which the insights are displayed. In this example the sleep score insights interface 9450 includes a diary insights section 9454, illustrating the effects of user entered activity on sleep score, with an accompanying text summary of the diary insights 9456. Through these insights, the user is informed of quantified effects on sleep resulting from her activity. In this example, the sleep score insights interface 9450 further includes a menstrual cycle insights section 9458, illustrating the effects of menstrual cycle phase on sleep score, with an accompanying text summary of the menstrual cycle insights 9460.

In this example the user can side scroll from the sleep score insights interface 9450, or select mood button 9312 in the home interface 9300, to display a mood insights interface 9470, as shown in FIG. 12P. The mood insights interface 9470 includes a graphical representation of the effect of sleep score on the user's mood—in this example mood vs sleep score scatter plot 9472—with an accompanying text summary of the mood insights 9474 (for example observing that the user's reported mood is better than average when her sleep score is above a particular threshold). In this example, the mood insights interface 9470 further includes a menstrual cycle mood insights section 9476, illustrating the effects of menstrual cycle phase on reported mood, with an accompanying text summary of the menstrual cycle mood insights 9478.

In this example the user can side scroll from the sleep score insights interface 9450 or mood insights interface 9470, or select focus button 9314 in the home interface 9300, to display a focus insights interface 9480, as shown in FIG. 12Q. The focus insights interface 9480 includes a graphical representation of the effect of sleep score on the user's focus—in this example focus vs sleep score scatter plot 9482—with an accompanying text summary of the focus insights 9484 (for example observing that the user's focus increases by a certain amount for a particular increase in sleep score). In this example, the focus insights interface 9480 further includes a menstrual cycle focus insights section 9486, illustrating the effects of menstrual cycle phase on reported mood, with an accompanying text summary of the menstrual cycle focus insights 9488.

### 5.12.2.1 Cognitive Behavioural Therapy

Cognitive behavioural therapy (CBT) is a form of psychological methodology for treating conditions, based on several principals like the ways of thinking, learning contributing behaviours & feelings, learning patterns, learning better ways of coping, reliving a user's symptoms and becoming more effective in their lives.

Cognitive behavioural therapy (CBT) emphasises the development of individuals to be their own mentors through exercises in the session as well to develop coping skills in which individuals learn to change their own thinking, problematic emotions, and behaviour. CBT provides insights of the person's current life with their known history and focuses primarily on more effective ways of coping in their life.

Cognitive behavioural therapy for Insomnia (CBT-i) is an effective compact, structured and evidence based insomnia treatment helping people suffering from insomnia to fall asleep faster, stay asleep and feel comfort during the day.

Cognitive behavioural therapy for Insomnia (CBT-i) focuses on exploring the connections between the ways of thinking, learning contributing factors and how we sleep. Cognitive behavioural therapy for Insomnia (CBT-i) provides insights using sleep diaries, sleep driving factors, circadian rhythm, Stimulus control, sleep hygiene and sleep restriction.

### 5.12.3 User Data Management and Access

According to aspects of the present technology, the system 9000 may be configured to make the data collected accessible to other parties subject to privacy controls, for example healthcare providers, research institutes, and healthcare companies. FIG. 13A(A)/13A(B) illustrates a user management interface 9500 displaying user record summaries 9502. Each user record summary may include for example: user ID, user name, menstrual cycle status (e.g. indicating whether the current menstrual cycle status is normal, or an irregularity is detected), sleep score, an indicator of use of the recommendations (e.g. on a percentage basis), last date of data synchronisation, last date of contact with the user.

In examples, selection of a user record summary may display a patient detail interface 9510 as shown in FIG. 13B(A)/13B(B). The patient detail interface 9510 acts as a dashboard providing an overview of the user's health data, in this example including: a sleep section 9512 (e.g. providing details of the user's sleep score over time, sleep state composition and duration, and breathing pauses), menstrual cycle section 9514 (e.g. providing details around cycle length and variability), heart rate section 9516 and recommendation use section 9518 (e.g. indicating the user's history of utilising the personalised recommendations).

### 5.12.4 Virtual Sleep Care

According to aspects of the present technology, there is provided a virtual sleep pathway for women. In examples a web portal 9600, as shown in FIG. 14, is provided to enable access to such a virtual sleep pathway. In examples the virtual sleep pathway may comprise one or more of: sleep assessment/screening services; on-demand telehealth visits with licensed practitioners who are attuned to female sleep issues and who can diagnose sleep disorders and/or provide CBT-i or prescribe OSA therapy; a curated selection of sleep solutions (e.g. wellness products and medical devices) designed for women's unique sleep needs.

FIG. 15 illustrates an exemplary virtual sleep pathway 9700. In a first aspect, a user engages directly with the virtual sleep pathway 9700 via a direct entry point 9702—for example engagement with search engine results or advertising, or social media advertising, or direct traffic to a web site. In this example, the user is directed to a screening stage 9704.

In the screening stage 9704, user inputs are received in response to a questionnaire. In examples, the user inputs include one or more of: hormonal status, information regarding diet and dietary habits, medication, supplements, menstrual cycle information, level of tiredness/fatigue, and exercise and healthy habit information (e.g. meditation, or bedtime habits).

In examples the questionnaire may include women-specific questions such as those outlined in the table below.

| Question | Selectable options |
|---|---|
| Does any of the | Pregnancy |
| following apply to | Lactation |
| you? These factors | Polycystic Ovarian Syndrome (PCOS) |
| may influence your | Menstrual related disorders (e.g. heavy menstrual |
| sleep. | bleeding, no menstrual bleeding, irregular menstrual |
| | bleeding, painful menstrual periods, premenstrual |
| | syndrome, premenstrual dysphonic disorder) |
| | Perimenopause (disable menopause selection if |
| | perimenopause is selected, either/or only) |
| | Menopause (disable perimenopause selection if |
| | menopause is selected, either/or only) |
| | None of the above |
| Do you use any | Pill |
| contraceptives? | Patch |
| Some contraceptives | Hormonal IUD |
| may influence your | Implant |
| sleep. | Intravaginal ring |
| | Injection |
| | Other (please specify) |
| | None of the above |
| What is your | <21 days |
| average cycle | 21-35 days |
| length? | >35 days |
| | No cycles |

In examples the menstrual cycle information may be determined using information obtained by one or more sensors, as described above. Additionally, or alternatively, the information may be input by the user.

In examples, one or more of the more inputs relating to the user's hormonal status may be obtained through biological testing—e.g. home or lab testing. For example, the inputs may include one or more of: glucagon, epinephrine (adrenaline), norepinephrine, cortisol, and growth hormone.

On receiving the user inputs, the information gathered is analysed to provide individualised insights and/or recommendations. In examples, one or more algorithms may be executed to analyse the information. In examples the information may be processed by an artificial intelligence model to provide the individualised insights and/or recommendations.

In the example illustrated, the screening stage 9704 is configured to determine an appropriate pathway for the user based at least in part on whether the user is likely to be impacted by male or female hormonal status. In alternative examples, the screening stage 9704 may assume the user is subject to female hormonal issues. Users identified as having female hormonal status are directed to a woman's pathway 9706, for example via the web portal 9600 of FIG. 14.

In examples, users identified as having a male hormonal status are directed to a gender agnostic pathway 9708—for example via communications and landing pages exemplified by the “Awaken your best” campaign by ResMed. In examples, users having female hormonal status but a higher risk of a respiratory disorder such as Obstructive Sleep Apnea (OSA) may be directed to a gender agnostic pathway 9708 which is more focused on such respiratory disorders.

Having considered the insights and/or recommendations, the user may select an assessment pathway focusing on respiratory disorders. In examples, a sleep respiratory disorder diagnosis stage 9710 may assess the user for a sleep respiratory disorder such as OSA—for example using the ResMed onesleeptest™ home sleep test, or myNight™ snoring and sleep quality application. In the event of the user being diagnosed with OSA, a CPAP trial stage 9712 may be undertaken.

In the event of the sleep respiratory disorder diagnosis stage 9710 indicating the user as being OSA-negative, but having symptoms indicative of insomnia, the user may be directed to an insomnia treatment stage 9714. Similarly, in the event of poor CPAP compliance during the CPAP trial stage 9712, with symptoms indicative of insomnia, the user may be directed to the insomnia treatment stage 9714. By way of example, objective sleep data, and factors that may influenced sleep is collected simultaneously (e.g. via a sleep and lifestyle diary). An algorithm will analyse and use the data to predict future events of problems falling asleep and/or staying asleep. In the insomnia treatment stage 9714, the user may undertake Cognitive behavioural therapy for Insomnia (CBT-i). In examples, the CBT-i may be delivered virtually.

In examples, the user may elect to circumvent the sleep respiratory disorder diagnosis stage 9710 and progress directly to the insomnia treatment stage 9714. In examples, the user may engage with their health provider (e.g. general practitioner) to discuss insomnia and develop a mental health care plan (MHCP) at MHCP stage 9716. In examples, the MHCP stage 9716 may be the user's entry point to the virtual sleep pathway 9700. Alternatively, the user may be directed to the MHCP stage 9716 from the woman's pathway 9706—for example in order to gain access to public health funding for subsequent treatment. Having developed a MHCP, a referral stage 9718 engages the user with a specialist for participation in the insomnia treatment stage 9714.

FIG. 16 illustrates an exemplary method 9800 for prediction of sleep related issues and provision of insights and/or recommendations in response to same. In a first step 9802 the user input in response to a data collection phase (for example the screening stage 9704) is received. In a second step 9804 the data is assessed for factors indicative of diet affecting the user's sleep. For example, one or more user inputs in response to the questionnaire may include key words indicative of dietary factors impacting sleep. For example, the one or more user inputs may indicate consumption of food or beverages containing substances such as alcohol and/or caffeine. As another example, one or more user inputs in response to the questionnaire may indicate the intake of substances (e.g. nicotine) which may impact sleep. In the event of such issues being identified, recommendations for diet with a view to improving sleep may be delivered in third step 9806.

In a fourth step 9808 the data is assessed for indications of medication and/or supplements affecting the user's sleep. For example, one or more user inputs in response to the questionnaire may include key words indicative of medication and/or supplements impacting sleep. For example the one or more user inputs may indicate one or more of: feeling tired/groggy/lethargic during the day, feelings of not having sufficient sleep due to dreams, having headaches, and not having much energy. In examples, a recommendation to consult with a specialist to evaluate medication and/or supplements may be provided in fifth step 9810.

In a sixth step 9812 the data is assessed for indications of hormonal status, in particular menstrual cycle, impacting the user's sleep. For example, the data may indicate one or more of: contraceptive use, pregnancy, lactation, polycystic ovary syndrome (PCOS), perimenopause, and menopause. In response to a determination of this being the case, insights regarding the menstrual cycle, hormonal fluctuations, menopause stages, and their impact on sleep health may be delivered to the user in seventh step 9814.

In an eighth step 9816 the data is assessed for indications of stress affecting the user's sleep. Such indicators may include, for example, one or more of: feeling tired/lethargic during the day, experiencing headache during the day, less total sleeping time, feeling anxious and/or depressed. In examples, in a ninth step 9818 recommendations for relieving stress and/or insights regarding the impact of stress on sleep may be provided in response to user inputs indicative of stress.

In examples, certain data may be used in more than one of the assessments outlined above.

In a tenth step 9820 the user inputs are holistically analysed to predict sleep related issues for the user, and insights and/or recommendations delivered to the user in eleventh step 9822. For example, such recommendations may include sleeping time adjustment, introduction of relaxation activities (e.g. yoga, and/or meditation), guidance regarding avoidance of alcohol and/or food impacting sleep after certain times.

### 5.13 Glossary

For the purposes of the present technology disclosure, in certain forms of the present technology, one or more of the following definitions may apply. In other forms of the present technology, alternative definitions may apply.

### 5.13.1 General

Air: In certain forms of the present technology, air may be taken to mean atmospheric air, and in other forms of the present technology air may be taken to mean some other combination of breathable gases, e.g. atmospheric air enriched with oxygen.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of Sleep Disordered Breathing (SDB) events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Patient: A person, whether or not they are suffering from a respiratory condition.

Pressure: Force per unit area. Pressure may be expressed in a range of units, including cmH₂O, g-f/cm2 and hectopascal. 1 cmH₂O is equal to 1 g-f/cm2 and is approximately 0.98 hectopascal (1 hectopascal=100 Pa=100 N/m2=1 millibar˜0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy (RPT): The application of a supply of air to an entrance to the airways at a treatment pressure that is typically positive with respect to atmosphere.

Ventilator: A mechanical device that provides pressure support to a patient to perform some or all of the work of breathing.

### 5.13.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

(inhalation) Time (Ti): The duration of the inspiratory portion of the respiratory flow rate waveform.

(exhalation) Time (Te): The duration of the expiratory portion of the respiratory flow rate waveform.

(total) Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.13.3 Ventilation

Adaptive Servo-Ventilator (ASV): A servo-ventilator that has a changeable, rather than fixed target ventilation. The changeable target ventilation may be learned from some characteristic of the patient, for example, a respiratory characteristic of the patient.

Backup rate: A parameter of a ventilator that establishes the minimum breathing rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired interface pressure which the ventilator will attempt to achieve at a given time.

End expiratory pressure (EEP): Desired interface pressure which the ventilator will attempt to achieve at the end of the expiratory portion of the breath. If the pressure waveform template Π(Φ) is zero-valued at the end of expiration, i.e. Π(Φ)=0 when Φ=1, the EEP is equal to the EPAP.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the respiratory portion of the breathing cycle by the patient's efforts.

### 5.14 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A method including:
receiving sleep related data for a user from one or more sensors;
receiving one or more inputs relating to the user's hormonal status;
determining one or more recommendations and/or insights to help improve the user's sleep; and
presenting the one or more recommendations and/or insights to the user.

2. The method of claim 1, wherein the one or more sensors include one or more of: heart rate sensors, oxygen level sensors, ECG sensors, pulse rate sensors, accelerometers, gyroscopes, GPS receivers, sound sensor and/or microphone, non-contact sensing device, and an optical sensor of a wearable sensor device configured to measure a blood volume pulse of the user.

3. The method of claim 1, wherein the one or more inputs relating to the user's hormonal status are user inputs.

4. The method of claim 3, wherein the one or more user inputs relating to the user's hormonal status include one or more of: level of tiredness/fatigue, instances of localised pain; period characteristics period timing characteristics of current or previous cycle, medication impacting menstrual cycle, and/or other factors including instances of hot flashes, nausea, constipation, acne, mood, and/or appetite.

5. The method of claim 3, wherein the one or more user inputs are received in response to a questionnaire.

6. The method of claim 1, wherein one or more of the inputs relating to the user's hormonal status are determined using one or more characteristics of the user obtained using one or more sensors.

7. The method of claim 1, wherein one or more of the inputs relating to the user's hormonal status comprise one or more of: glucagon, epinephrine (adrenaline), norepinephrine, cortisol, and growth hormone.

8. The method of claim 1, including receiving one or more inputs relating to the user's exercise habits.

9. The method of claim 8, wherein the one or more inputs relating to the user's exercise habits may be received via one or more of: in response to the questionnaire, and one or more exercise applications.

10. The method of claim 1, wherein the one or more recommendations comprise one or more of: lifestyle changes, sleep hygiene, Auto Sleep Diary, ideal bed time, sleep programs, relaxation exercises, educational articles, consulting a sleep consultant, consulting a medical professional, and/or screening for a sleep related disorder.

11. The method of claim 1, wherein the one or more insights comprise: ovulation period, hormone fluctuations, effect of hormone fluctuations on sleep, correlation between a menstrual cycle phase and an indicator of sleep of the user, correlation between an indicator of sleep and user entered factors, an indicator of readiness, and/or guidance on factors which appear to be impacting the user's sleep and/or readiness.

12. The method of claim 1, including predicting the user suffering from problems with falling or staying asleep based on monitoring of one or more factors over an extended period of time.

13. The method of claim 12, wherein the extended period of time is one of: at least seven days, and at least fourteen days.

14. The method of claim 11, wherein the one or more factors include one or more of: caffeine intake, sleep start time, diet, supplements, medications, symptoms, menstrual cycle phase, average cycle length, contraceptive use, pregnancy, lactation, Polycystic Ovarian Syndrome (PCOS), menstrual related disorders, perimenopause, menopause, improving sleep habits, life experiences, and stress relief activity.

15. A method including:
receiving sleep related data for a user from one or more sensors;
receiving one or more inputs relating to the user's hormonal status; and
presenting information on the user's hormonal status along with sleep data on a user interface.

16. A virtual sleep clinic comprising one or more of: sleep assessment services; on-demand telehealth visits with a licensed practitioner who is attuned to female sleep issues and who can diagnose sleep disorders, provide behavioural therapy, and/or prescribe therapy; and provision of sleep solutions designed for female sleep needs, wherein the virtual sleep clinic is configured to facilitate performance of a method including:
receiving sleep related data for a user from one or more sensors;
receiving one or more inputs relating to the user's hormonal status;
determining one or more recommendations and/or insights to help improve the user's sleep; and
presenting the one or more recommendations and/or insights to the user.

17. The virtual sleep clinic of claim 16, wherein the virtual sleep clinic is configured to facilitate performance of a screening stage to determine a sleep assessment pathway for a user.

18. The virtual sleep clinic of claim 17, wherein the screening stage identifies whether the user is likely to be impacted by male or female hormonal status, and directs the user to a sleep pathway based at least in part on this identification.

19. The virtual sleep clinic of claim 17, wherein a first sleep assessment pathway is directed to sleep respiratory conditions, and a second sleep assessment pathway is directed to problems with falling or staying asleep.

20. A computer program product comprising instructions which, when executed by a computer, cause the computer to carry out a method including:
receiving sleep related data for a user from one or more sensors;
receiving one or more inputs relating to the user's hormonal status;
determining one or more recommendations and/or insights to help improve the user's sleep; and
presenting the one or more recommendations and/or insights to the user.
